(19) United States
(12) Patent Application Publication
(10) Pub. No.: US 2022/0323791 A1
(43) Pub. Date: Oct. 13, 2022

(54) FLASH RADIOTHERAPY SYSTEMS AND METHODS OF USE

(71) Applicant: The New York Proton Center, New York, NY (US)

(72) Inventors: Minglei Kang, New York, NY (US); Shouyi Wei, New York, NY (US); J. Isabelle Choi, New York, NY (US); Haibo Lin, New York, NY (US); Charles B. Simone, New York, NY (US)

(73) Assignee: The New York Proton Center, New York, NY (US)

(21) Appl. No.: 17/719,801

(22) Filed: Apr. 13, 2022

Related U.S. Application Data
(60) Provisional application No. 63/174,461, filed on Apr. 13, 2021.

(51) Int. Cl. A61N 5/10

(52) U.S. Cl. CPC A61N 5/1048; A61N 5/1031; A61N 2005/1087

(57) ABSTRACT

Disclosed herein are cancer treatment methods.

### INCORPORATION BY REFERENCE OF RELATED APPLICATION

This application claims priority to U.S. Provisional Patent Application No. 63/174,461 filed Apr. 13, 2021, which is incorporated by reference herein in its entirety.

### TECHNICAL FIELD

The present disclosure generally relates to systems and methods for providing radiotherapy.

### BACKGROUND

Radiotherapy is a key treatment for both curative and palliative cancer care. However, radiotherapy is limited by radiation-induced toxicities. Pre-clinical studies have shown that irradiation at dose rates far exceeding those currently used in clinical contexts reduces radiation-induced toxicities while maintaining an equivalent tumor response. This is known as the FLASH effect. The mechanism responsible for reduced tissue toxicity following FLASH radiotherapy (FLASH-RT) is still undetermined; multiple hypotheses have been suggested by linking the high dose rate to rapid oxygen depletion, immune response, reduction of peroxyl radical lifetime, preservation of normal tissue stem cells, etc.

Recently, there has been growing interest in using proton therapy to conduct FLASH-RT translational research. Transmission/shoot-through beams have been proposed for FLASH proton treatment planning.

### SUMMARY

In a first embodiment, a method for supplying a field of ionizing radiation to a target tissue is provided, which includes providing an ionizing radiation, forming at least two fields of shifted and compensated ionizing radiation by shifting the range of the ionizing radiation by passing the ionizing radiation through an adjustable range shifter, so that the Bragg peak of the ionizing radiation coincides with the target tissue; and compensating the range of the ionizing radiation by passing the ionizing radiation through an adjustable range compensator, so that the Bragg peak of the ionizing radiation coincides with the target tissue; and directing to the target tissue at least two fields of the shifted and compensated ionizing radiation to provide a uniform dose distribution across a target volume.

The first embodiment may include administration of a dose rate of at least 40 Gy/s. The first embodiment may include administration of shifted and compensated ionizing radiation that does not substantially extend proximally beyond a distal edge of the target location. The embodiment may include compensated ionizing radiation composed of protons, helium, carbon, argon or neon. The embodiment may include compensated ionizing radiation composed of protons.

In some embodiments the target location is cancerous tissue. In some embodiments the range shifter includes multiple plates that reduce the range of the ionizing radiation, and said combinations of the range shifters are calculated by applying parameters determined using an inverse-planning optimization protocol, wherein said parameters comprise the number and location of the plates through which the ionizing radiation is transmitted. In embodiments the range compensator contours are calculated by applying parameters determined using an inverse-planning optimization protocol. In some embodiments, inverse-planning optimization determines the distribution parameters of the ionizing radiation, and/or determines the weighting parameters of the ionizing radiation. In some embodiments, the shifted and compensated ionizing radiation includes three, four, or five fields of the shifted and compensated ionizing radiation.

A further embodiment can include a system for administering at least two fields of shifted and compensated ionizing radiation to a target tissue, the system including an ionizing radiation source configured to produce a charged particle beam; a universal range shifter adjusted to shift the range of the charged particle beam so that the Bragg peak of the charged particle beam coincides with the target tissue; and a range compensator adjusted to compensate the range of the charged particle beam so that the Bragg peak of the ionizing radiation coincides with the contour of the target tissue. The embodiment can include administration of the fields in a dose rate of at least 40 Gy/s. In some embodiments the fields do not substantially extend proximally beyond a distal edge of the target tissue. In embodiments the target tissue comprises a neoplasm or benign tumor. In embodiments the range shifter includes multiple plates that reduce the range of the ionizing radiation, and combinations of the range shifters are calculated by applying parameters determined using an inverse-planning optimization protocol, wherein said parameters include the number and location of the plates through which the ionizing radiation is transmitted. In some embodiments the range compensator contours are calculated by applying parameters determined using an inverse-planning optimization protocol. In embodiments, the at least two fields of the shifted and compensated ionizing radiation comprises three fields, or four fields, or five fields.

A further embodiment comprises a system for producing at least two shifted, compensated fields of particle beams at a dose rate of at least 40 Gy/s, said system including an ionizing radiation source configured to produce a particle beam; a universal range shifter configured to adjustably shift the range of the particle beam; and a range compensator configured to adjustably compensate the range of the particle beam. In embodiments the particle beams include protons, helium, carbon, argon, or neon.

Another embodiment comprises a method of treating a target tissue, including diagnosing a target tissue; mapping the target tissue; developing a radiotherapy treatment plan to administer an effective amount of shifted and compensated ionizing radiation to the target tissue; and shifting and compensating an ionizing radiation using a system including an ionizing radiation source configured to produce a particle beam; a universal range shifter adjusted to shift the range of the proton/particle beam so that the Bragg peak of the particle beam coincides with the target tissue; a range compensator adjusted to compensate the range of the particle beam so that the Bragg peak of the particle beam coincides with the contour of the target tissue; and then administering the particle beam to the target tissue. In embodiments the particle beam is applied in a dose rate of at least 40 Gy/s. In embodiments the target tissue includes a neoplasm or benign tumor. In embodiments the particle beam does not substantially extend proximally beyond a distal edge of the neoplasm or benign tumor. In embodiments wherein the range shifter includes multiple plates that reduce the range of the ionizing radiation, and the range shifter plate positioning is determined by applying parameters determined using an inverse-planning optimization protocol, wherein said parameters comprise the number and location of the plates through which the ionizing radiation is transmitted. In embodiments, the shape of said range compensator is calculated by applying parameters determined using an inverse-planning optimization protocol. In embodiments, the at least two fields of the shifted and compensated ionizing radiation comprises three fields, or four fields, or five fields. In embodiments the particle beams include protons, helium, carbon, argon, or neon.

Another embodiment comprises a method of adjusting a proton therapy device including receiving a treatment plan designed to apply FLASH-RT to a target location, wherein said treatment plan comprises a target location, a three-dimensional target shape, a number of treatment fields, and target dose rate of at least 40 Gy/s; and modifying the energy or range of the proton therapy device using a range shifter and a range compensator, wherein said range shifter comprises multiple plates that reduce the range of the ionizing radiation, wherein said modifying comprises use of an inverse-planning protocol to determine range shifter and range compensator parameters so that the Bragg peak of the energy output of the proton therapy device coincides with a target tissue, and wherein said parameters comprise the number and location of the plates through which the energy or range of the proton therapy device is transmitted. In embodiments the energy output of the proton therapy device does not substantially extend proximally beyond a distal edge of the cancerous tissue.

A further embodiment includes a universal range shifter including six polycarbonate plastic plates, and capable of producing a range shifted ionizing radiation by reducing the range of an ionizing radiation between 0 cm to 34 cm in 1 cm increments. In embodiments the ionizing radiation includes protons, helium, carbon, argon, or neon. In embodiments the thickness of the polycarbonate plastic plates is 1, 2, 3, 7, 7, and 14 cm WET.

A further embodiment includes radiotherapy treatment device including a universal range shifter, the universal range shifter including six polycarbonate plastic plates, and capable of producing a range shifted ionizing radiation by reducing the range of an ionizing radiation between 0 cm to 34 cm in 1 cm increments. In embodiments the ionizing radiation includes protons or other ions. In an embodiment the thickness of said polycarbonate plastic plates is 1, 2, 3, 7, 7, and 14 cm WET.

A further embodiment includes a method of reducing the range of an ionizing radiation by between 0 cm and 34 cm in 1 cm increments, said method comprising passing the ionizing radiation through a universal range shifter comprising six polycarbonate plastic plates. In embodiments the ionizing radiation includes protons, helium, carbon, argon, or neon. In embodiments the thickness of the polycarbonate plastic plates is 1, 2, 3, 7, 7, and 14 cm WET.

Another embodiment includes a method for treating a cancerous tissue comprising; providing an ionizing radiation transmission beam with a dose rate of at least 40 Gy/s; adjusting the energy or range of the ionizing radiation transmission beam such that the Bragg peak of the beam coincides with a point between 3 mm and 5 mm from an edge of the cancerous tissue; and applying the ionizing radiation transmission beam to the cancerous tissue. In an embodiment the ionizing radiation transmission beam includes protons, helium, carbon, argon, or neon.

### BRIEF DESCRIPTION OF THE FIGURES

FIG. 1 shows a schematic diagram of non-transmission FLASH intensity-modulated particle therapy (IMPT) planning using universal range shifters (URS) and range compensators (RC). The URS and RC are placed in the beam path for illustration purpose only; they can be placed in various order depending on treatment goals.

FIG. 2 shows that an example of spot distribution and weight optimization can effectively improve the plan quality and FLASH-RT dose rate distribution.

FIG. 3 shows transmission ((a) & (d)) vs. Bragg peak ((b) & (e)) planning using 250 MeV proton beams for C-shape target in a water phantom.

FIG. 4 shows the dose comparisons between transmission and Bragg peak plans for three selected lung patients using the same beam arrangement. The right and middle columns represent transmission and Bragg peak plans, respectively.

FIG. 5 shows dose rate comparisons between transmission (the left side images) and Bragg peak (the middle images) plans using the same beam arrangement.

FIG. 6 upper view: the single spot (1000 MU/spot) 2-D dose rate distribution for 250 MeV proton beam at central axis plane evolves in water phantom with air gaps of 5, 15, and 25 cm, respectively; lower view: the spot dose rate at the central axis (the three sections represent the 20 cm transport in water, air gaps, and the residual range in water).

FIG. 7 shows an example illustrating beam angle optimization. (a) and (b) are the 2D dose distribution using different fields and field angles, (c) and (d) are the DVH, and DRVH comparison, (e) is the V_{40Gy/s} dose rate coverage vs. OAR doses from the low to high dose regions. The left side lung and most of the heart are completely spared using a beam arrangement shown by (b).

FIG. 8 shows an exemplary Range Compensator (RC).

FIG. 9 shows the dosimetric comparison between Bragg peak and conventional IMPT plans for 10 liver cancer patients. (a) Liver-GTV Dₘₑₐₙ, (b) heart D_{0.5cc}, (c) chest wall D_{2cc}, and (d) CTV Dₘₐₓ for SBRT, Bragg-400MU, and Bragg-800MU. n.s. represents that the results are statistically non-significant (p≥0.05). The interquartile (25-75th percentile) is denoted by the ends of the box, the median is represented by a horizontal line inside of the box, and the highest and lowest values are denoted by two lines outside of the box. The diamond marker indicates data points beyond the 25-75^{th} percentile. Liver treatment planning studies demonstrated that the novel single-energy PBS delivery method can achieve a similar or equivalent plan quality compared to the conventional multi-energy proton PBS plans.

TABLE 1 shows dosimetry and dose rate coverage of V_{40Gy/s} comparison for transmission and Bragg peak IMPT plans for all six lung cases. The dosimetry comparison used RTOG 0915 metrics. Both dose and dose rate statistics used the averaged values for all six cases. The last row of the table represents the averaged V_{40Gy/s} for both target and OARs.

TABLE 2 shows how an exemplary URS's six range shifter plates can generate 34 different combinations to “pull back” or reduce the proton range between 0 cm to 34 cm with a step of 1 cm. Here, “1” represents that the plate will be moved into the beam path to pull back the proton beam range, and “0” means that plate won't be used to pull the range back.

TABLE 3 shows plan quality comparisons between Bragg Peak and conventional IMPT plans for 10 lung cancer patients. Lung treatment planning studies demonstrated that the novel single-energy PBS delivery method can achieve a similar or equivalent plan quality compared to the conventional multi-energy proton PBS plans. p1, p-values of the two-tailed student t-tests between BP-1200MU-2 ms vs IMPT-SBRT plans; p2, p-values of the two-tailed student t-tests between BP-300MU-0.5 ms vs IMPT-SBRT plans.

### DETAILED DESCRIPTION

FLASH-RT improves patient outcomes but requires specialized hardware and expertise. Further, current transmission/shoot-through plans do not utilize Bragg peaks for dose delivery, which results in unnecessary irradiation exposure to normal tissues distal to the target volume. In the present disclosure “distal” to refers to further along a beam path, as opposed to “anatomically” distal.

Single-energy pencil beam scanning (PBS) delivery methods disclosed herein can achieve a similar or equivalent plan quality compared to conventional multi-energy proton PBS plans. By eliminating the expensive energy selection systems and beam focusing systems, the proton treatment cost will be significantly reduced and make PBS FLASH-RT more affordable for the public. Use of a single energy-layer proton beam from a cyclotron for conformal conventional dose rate/FLASH-RT can be a promising solution for future proton system design.

In contrast to prior methods, disclosed embodiments comprise commercially-available equipment modified with additional components to utilize the Bragg peak—a pronounced peak on the Bragg curve which plots the energy loss of ionizing radiation during its travel through matter. For protons, α-rays, and other ion rays, the peak occurs immediately before the particles come to rest. It will be appreciated that the Bragg curve for these sorts of particles are qualitatively different than those for x-rays or other types of electromagnetic radiation.

Disclosed methods and systems include FLASH-RT tumor treatment modalities that modify currently-available proton and other charged heavy particles (for example helium, carbon, argon and neon) systems utilizing methods based on an inverse optimization algorithm that requires minimal hardware modification to utilize the proton beam Bragg peak region to treat tumors. The Bragg peak is a pronounced peak on the Bragg curve which plots the energy loss of ionizing radiation during its travel through matter.

The Bragg peak can be identified using a graphic representation of the energy of certain charged particles such as protons-energy lost by certain charged particles is inversely proportional to the square of their velocity, thus the graphed peak occurs just before the particle comes to a complete stop. By correlating the Bragg peak with the target tissue, disclosed methods and systems avoid the “exit dose” transmitted far beyond a target tissue and, therefore, spare tissue adjacent to target areas. The Bragg peak can be calculated using commercially available software.

Disclosed systems include adjustable, universal range shifters (URS) comprising materials such as plastic plates that “pull back” or reduce the energy of ionizing radiation beams, and thus reduce the range of the beams. By providing a number of “plates” of various thicknesses at discrete distances in the beam path, disclosed systems allow a user to accurately and reproducibly reduce the range of the beam to a target depth in tissue using ionizing radiation sources that would otherwise be unsuitable for FLASH-RT utilizing the Bragg peak of the particles comprising the ionizing radiation.

Disclosed systems include range compensators (RC) comprising materials that further modify the range of ionizing radiation beams, and thus tailor the penetration depths of the beams to deliver conformable radiation to target tissue. For example, disclosed RCs comprise a three-dimensional contour to finely adjust the range of the range-shifted ionizing radiation (an exemplary RC is shown in FIG. 8). By providing range compensators of various thicknesses in beam direction and covering the target tissue laterally using scanned discrete proton spots for the determination of the optimal 3-D shape, disclosed systems allow a user to accurately and reproducibly conform the beam to the distal target shape using ionizing radiation sources that would other wise be unsuitable for FLASH-RT using the Bragg peak of the particles comprising the ionizing radiation. The RC can finely adjust the ionizing radiation before, during, or after the radiation is range-shifted.

Disclosed methods comprise adjusting the range of an energy beam, for example a proton beam, and compensating proton ranges to target an area of target tissue such that the Bragg peak of the beam coincides with the target tissue, for example the distal area of target tissue, or in some cases immediately beyond the distal edge of the target tissue, thus reducing or eliminating the “exit” dose of proton beams that extend beyond the target, while still preserving FLASH-RT effectiveness. As a result of the rapid drop-off in particle energy at the penetration range reflected by the Bragg peak, the majority of the particle energy can be limited to the target tissue.

In intensity-modulated radiation therapy (IMRT) such as intensity-modulated particle therapy (IMPT), the beam intensity is varied across each treatment region (target) in a patient. Depending on the treatment modality, parameters available for intensity modulation include beam shaping (collimation), beam weighting (spot scanning), and angle of incidence (beam geometry). These degrees of freedom lead to an effectively infinite number of potential treatment plans. Therefore, consistently and efficiently generating and evaluating high-quality treatment plans relies on the use of computing systems.

An inverse planning tool was developed to optimize IMPT using a single-energy layer for FLASH-RT planning. Inverse planning is the process by which the intensity distribution of each beam employed in a treatment plan is determined such that the resultant dose distribution can best meet the criteria specified by the planner. In inverse planning, a radiation oncologist defines a patient's critical organs and tumor volume, after which they also determine target doses and importance factors for each. Then, a planner runs an optimization program to find the treatment plan that best matches all the input criteria. Thus, inverse planning uses the optimizer to solve the Inverse problem as set up by the planner. The range pulling-back values and physical compensator contours may be calculated to stop single-energy proton beams at the distal edge of the target.

The spot map and weights of each field were optimized to achieve a sufficient dose rate using proton beam Bragg peaks. Treatments of both “phantom” (model) and patients were planned using disclosed methods and transmission techniques to assess dosimetry and dose rate characteristics.

### Definitions

“Administration,” or “to administer” means the step of giving (i.e. administering) a treatment to a subject.

“Bragg Curve” means a graph of the energy loss rate of a particle, or Linear Energy Transfer (LET), as a function of the distance through a stopping medium. The energy loss is characterized primarily by the square of the nuclear charge, Z, and the inverse square of the projectile velocity, β. This gives the Bragg Curve its shape, “peaking” (and thus showing the range at which a particle is releasing most of its energy) just before the projectile stops.

“Bragg Peak” is a pronounced peak on the Bragg Curve which plots the energy loss of ionizing radiation during its travel through matter. The Bragg peak identifies the range at which a particle releases most of its energy.

“Calculate” refers to the selection and adjustment of the range shifters and/or range compensator to determine the positioning and combinations of the plates to produce an ionizing radiation of the desired range.

“Cancerous tissue” means any neoplasm or benign tumor.

“FieId” means an area treated by an ionizing radiation beam at a particular angle. Radiotherapy treatment can be delivered using a single field or multiple fields at different angles.

“FLASH radiotherapy” or “FLASH-RT” is a radiotherapy treatment method that delivers hypofractionated and elevated radiation dose with ultra-high dose rates.

“Particle beams” refers to an ionizing radiation comprising protons or other heavy particles, for example helium, carbon, argon and neon.

“Patient” means a human or non-human subject receiving medical or veterinary care.

“Plate” as used herein refers to a URS or RC component (for example, polycarbonate plastic) through which an ionizing radiation is passed to lower the energy of the ionizing radiation or to reduce the penetration ranges, or to finely adjust the beam penetration ranges of the ionizing radiation.

“Range Compensator” or “RC” means hardware configured to finely adjust or reduce the the range of an ionizing radiation into a beam form suitable for administration for Bragg peak-based FLASH-RT as described herein. Range compensators finely adjust the range of the shifted ionizing radiation to account for the three-dimensional shape of the target tissue.

“Target tissue” refers to the tissue to be treated, for example cancerous tissue such as a cancerous tissue including neoplasms and benign tumors.

“Tuned” as used herein means to optimize the number of spots, weightings of spots, and locations of spots during inverse optimization to determine spot map to reach an optimal plan.

“Universal Range Shifter” or “URS” means hardware configured to adjust/reduce the range of an ionizing radiation range into a form suitable for administration for Bragg peak-based FLASH-RT as described herein.

Administration Systems

Some embodiments disclosed herein comprise administration systems, such as administration systems for administering FLASH-RT treatments. In embodiments, disclosed systems comprise a source of ionizing radiation, for example, a cyclotron or a synchrotron. In embodiments, the ionizing radiation source emits an ionizing radiation, for example protons, helium, carbon, argon and neon, or other heavy particles.

Some disclosed embodiments further comprise range shifters. For example, in disclosed embodiments, range shifters can comprise universal range shifters (URS), that can convert an ionizing radiation into fields of discrete range. In disclosed embodiments, the URS can comprise multiple plastic plates, for example transparent amorphous thermoplastic plates such as polycarbonate plastic plates, with varying thicknesses. In embodiments, the ranges of the separate fields can be shifted by differing amounts. For example, in an embodiment employing 5 fields for treatment, the five fields may comprise 1, 2, 3, 4, or 5 different range shifts, thus 1, 2, 3, 4, or 5 different URS plate combinations.

Some disclosed system embodiments further comprise range compensators (RC). For example, in disclosed embodiments, the RC can comprise at least one plastic plate with contours having various thicknesses, for example a solid, transparent amorphous thermoplastic plate such as a polycarbonate plastic plate. In embodiments, the plates can be of a density of, for example, 0.5 g/cm³, 0.6 g/cm³, 0.7 g/cm³, 0.8 g/cm³, 0.9 g/cm³, 1.0 g/cm³, 1.1 g/cm³, 1.2 g/cm³, 1.3 g/cm³, 1.4 g/cm³, 1.5 g/cm³, 1.6 g/cm³, or the like. In embodiments, the RC can further refine the range of the ionizing radiation that has been range-shifted with the URS.

In embodiments, the range of the separate fields can be altered by different RCs. For example, in an embodiment employing 5 fields for treatment, the five fields may comprise 1, 2, 3, 4, or 5 different RCs. An exemplary RC is shown in FIG. 8, with the 3-D “topography” of the RC clearly visible.

When combined with disclosed URS, RC, and inverse planning embodiments, this allows for the provision of Bragg peak-utilizing FLASH-RT treatments using conventional proton RT equipment, for example by modifying the beam range.

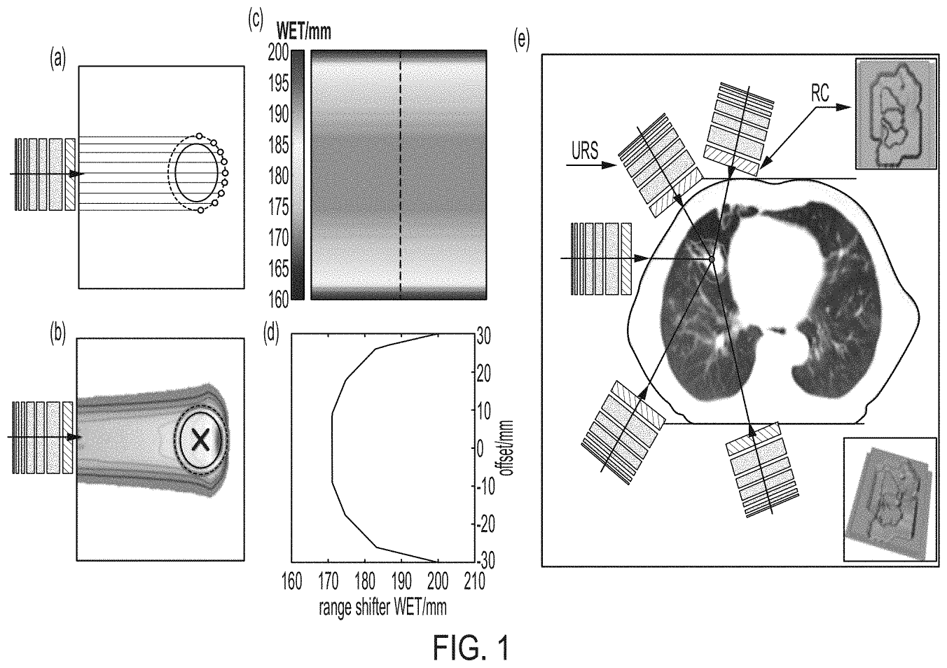

Some disclosed embodiments comprise inverse-planning to target the ionizing radiation. For example, in embodiments, ray tracing is used to calculate the range compensation. In embodiments, an energy beam is customized to generate the intensity-modulated spot map via the inverse planning platform. For example, as shown in FIG. 1(a), a uniform margin of, for example, 6-mm on the CTV can be used to contain the spot distribution in-depth direction. The 90% of dose falloff can be used as the proton range for spot map generation. The water equivalent thickness (WET) of each pencil beam proton radiographic track (denoted by WETi (x, y, z)) can be calculated by Eq. 1, and rsp(x, y, z) represent the relative stopping power (rsp) of each voxel of the 3D CT images.

The integral step in Eq. 1 can be accurately computed with a raytracing algorithm (Siddon R L. Prism representation: A 3D ray-tracing algorithm for radiotherapy applications. Phys. Med. Biol. 1985; 10.1088/0031-9155/30/8/005 30(8), 817-824). Each pencil beam range pulling-back or reduction can be calculated by Rᵢ, where R_{Eo}, is the range of the highest energy in water. Disclosed FLASH-RT Bragg peak treatment plans can employ a multi-field arrangement, for example a 5-field beam arrangement, and a multiple-field-optimization (MFO) method can be used to generate spot maps.

Equation 1:

WETᵢ(x,y,z)=∫₀^{depth}rsp(x,y,z)dlᵢ

Rᵢ=R_{E0}−WETᵢ(x,y,z) (1)

In embodiments, the total desired range compensation for each field can be achieved by using a URS and an RC. The thickness of the URS can vary, for example from 0 to 34 cm, which, with the assistance of the RC, enables the treatment of tumors at all depths.

In embodiments, the URS can comprise, for example, at least 1 plate, at least 2 plates, at least 3 plates, at least 4 plates, at least 5 plates, at least 6 plates, at least 7 plates, at least 8 plates, at least 9 plates, at least 10 plates, at least 11 plates, at least 12 plates, at least 13 plates, at least 14 plates, at least 15 plates, at least 16 plates, at least 17 plates, at least 18 plates, at least 19 plates, at least 20 plates, or more In one example system, six plates may be used to generate a range of desired depths (0-34 cm) which may be adequate for many purposes.

In embodiments, the thickness of the individual URS plates can be, for example, 1 cm water equivalent thickness (WET), 2 cm WET, 3 cm WET, 4 cm WET, 5 cm WET, 6 cm WET, 7 cm WET, 8 cm WET, 9 cm WET, 10 cm WET, 11 cm WET, 12 cm WET, 13 cm WET, 14 cm WET, 15 cm WET, 16 cm WET, 17 cm WET, 18 cm WET, 19 cm WET, 20 cm WET, 21 cm WET, 22 cm WET, 23 cm WET, 24 cm WET, 25 cm WET, 26 cm WET, 27 cm WET, 28 cm WET, 29 cm WET, 30 cm WET, or more.

In an embodiment, the URS comprises 6 polycarbonate plastic plates of thicknesses of 1, 2, 3, 7, 7, and 14 cm WET, generating 35 discrete range reduction increments with a depth resolution of 1 cm. The range plate combinations for 35 discrete range pulling-backs used in the study described in Example 1 are depicted in Table 2.

FIG. 1(e) shows the schematic of a URS system of 6 polycarbonate plastic plates of thicknesses of 1, 2, 3, 7, 7, and 14 cm WET used in Example 1, generating 35 discrete range pulling-backs with a depth resolution of 1 cm. Each range shifter plate was driven by a standalone step motor to move “in” and “out” of the beam path, and the “in” and “out” combination of the six plates is similar to a binary system that can generate the correct range pulling back. The range plate combinations for 35 discrete range pulling-backs are depicted in Table 2.

In embodiments, the thicker range shifters can be placed closer downstream, and the thinner range shifters are more upstream, a design consideration to minimize the transport distance of scattered proton beams to reduce spot size and preserve a high spot peak dose rate (SPDR). The desired proton ranges are achieved by moving the range shifter plates “in” and “out” of the beam path. The thickness of URS used in each beam path can be calculated using R_{URs} in Eq.2. The max thickness of RC can be determined by R_{c} in Eq. 2. Therefore, in embodiments, the total range pulling-back capacity can be between 0 and R_{E0} cm which can accommodate deep and superficial targets.

The range compensation Rᵢ of each proton trace under each field can be calculated and stored by, for example, a 3D data matrix. The data sets can be used to construct 3D printed compensators conveniently. As shown in FIG. 1(e), the RC is presented on the right upper and lower corner.

Equation 2:

f(x,y,z)=max(WETᵢ(x,y,z))−min(WETᵢ(x,y,z))

R_{C}=fcm and R_{URS}=(R_{E0}−f)cm (2)

Similar to the compensator design for scattering proton systems, a smearing method [Moyers M F, Miller D W, Bush D A, Slater J D. Methodologies and tools for proton beam design for lung tumors. Int J Radiat Oncol Biol Phys 2001; 49(5):1429-38.] can be used to design the RC to manage range uncertainties.

In embodiments, the minimal MU/spot or minimal treatment room beam current in nanoampere (nA) required for treatment can determine the dose rate of each energy layer, and the minimal MU/spot and dose rate are further optimized to reach the FLASH dose rate threshold. An algorithm can be used to generate an optimal spot map via two steps:
a. first, an initial minimal MU/spot threshold (w₀) can be used for inverse optimization and a dense spot map with a defined spot spacing can be generated;
b. second, the low weighting spots can be merged to new spots by applying both a distance threshold rₜ and a weighting factor of wₜ, in which the rₜ is a ratio of spot spacing. The wₜ is a factor-based minimal MU/spot requirement for FLASH dose rate. As shown in Eq.3, the weights can be combined as wₘ, and the spot coordinates are calculated based on their original coordinates and weighting fractions using Eq.4. The final spot location can be determined by applying the coordinate threshold rₜ, described using Eq. 5.

In some embodiments, there are at least two considerations for applying the second step to generate the final spot maps.
a. First, as the minimal MU/spot determines the SPDR of the layer, by merging the low weighting spots, a high SPDR can be achieved.
b. second, the low weighted spots are important to maintain a good plan quality as with conventional IMPT plan optimization. By merging lower weighted spots to the nearby ones, the spot distribution pattern is minimally changed, but better plan dosimetry distribution is achievable.

It will be appreciated that other planning steps and algorithms may also be employed.

In some disclosed embodiments, continuous optimization can be performed to fine-tune the spot weights to further improve target uniformity and OARs sparing. By iteratively applying the second step of spot map optimization (merging lower weighted spots to the nearby ones), the dose rate can be continuously improved.

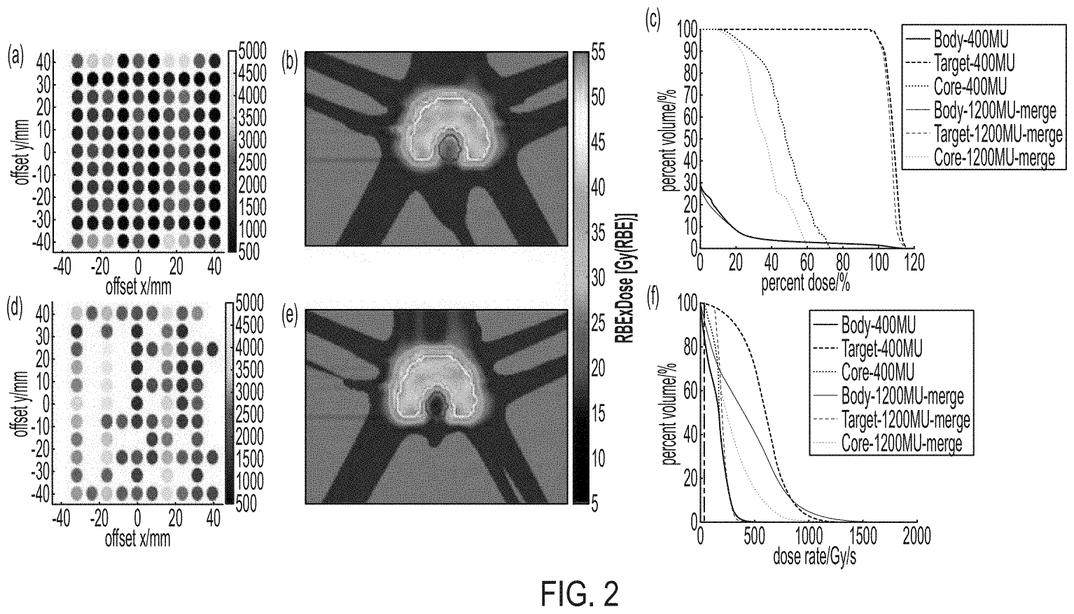

In embodiments, the efficacy of the spot map optimization can be tested using a C-shape target that surrounds a central avoidance core structure. As shown in FIG. 2, (a) is the spot map of one field using an initial 400 MU/spot threshold, (d) is the spot map after applying the spot map optimization process, (b) and (e) are the 2D dose distribution comparison for a selected slice, (c) and (f) are the DVH and DRVH comparison. By doing so, the low dose region is reduced, and the conformity improved, as can be seen from the 2D dose distribution, and the DVH of the core structure resultingly shifted towards the lower dose end substantially. The dose rates to body, target, and core structure can be increased, illustrated in (f).

$$\begin{matrix}Equations 3 , 4 , and 5 : & \\ {w}_{m} = {w}_{i} + {w}_{i + 1} & ( 3 )\end{matrix}$$

$$\vec{{r}_{m} ( x , y )} = \frac{{w}_{i}}{{w}_{m}} \vec{{r}_{i} ( x , y )} + \frac{{w}_{i + 1}}{{w}_{m}} \vec{{r}_{i + 1} ( x , y )} \qquad ( 4 )$$

$$\vec{{r}_{m} ( x , y )} = arg min \vert \vec{{r}_{m} ( x , y )} - \vec{{r}_{t} ( x , y )} \vert \qquad ( 5 )$$

FIG. 2 shows an example of spot distribution and weight optimization that can effectively improve the plan quality and FLASH-RT dose rate distribution. (a) and (d) the spot maps before and after the spot map optimization process; (b) and (e) the 2D dose distribution comparison; (c) and (f) the DVH and DRVH comparisons before and after spot map optimization. A dashed line from the DRVH marks the 40 Gy/s threshold.

In some embodiments, the multiple Coulomb scattering (MCS) between the protons and USR and RC can enlarge the spot divergence significantly. Equivalently, the scattering effects can also result in progressive shortening of the effective-SSD of the beam. At a shorter effective-SSD, proton fluence decreases more quickly due to a larger inverse square effect, and the spot size increases more rapidly.

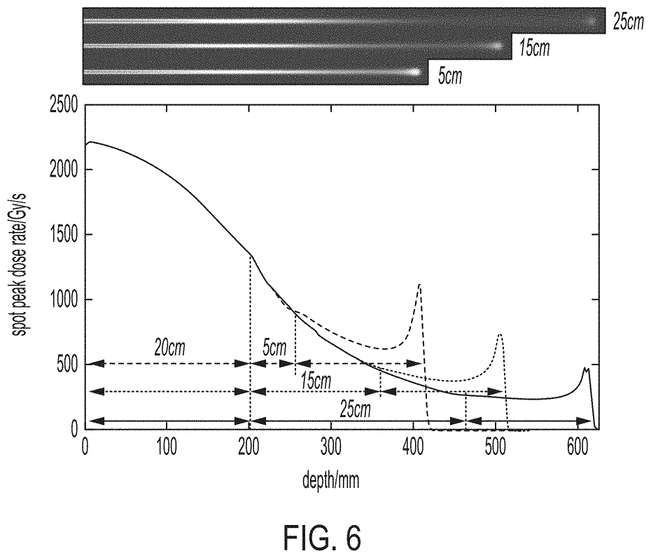

FIG. 6 illustrates the dose rate distribution for a 250 MeV single spot with 1000 Monitor Units (a measure of machine output from a clinical accelerator for radiation therapy; [MU]) in a water phantom. In Example 1, 5, 15, and 25 cm air gaps between the RC and the phantom surface were “mimicked” to calculate the spot dose rate at the central axis in water changing with air gaps. It was clear that the spot dose rate decreased when the air gap increased, and the central axis dose rate at the Bragg peak is reduced by a factor of ˜2 between 5 cm and 25 cm air gaps. During FLASH plan optimization, minimizing the air gap often plays an important role in maintaining proton fluence intensity and a smaller penumbra, which may be crucial for the OAR sparing. Meanwhile, a large spot size caused by MCS and a large air gap will significantly reduce the spot dose rate and the treatment field's mean dose rate. To achieve a higher spot dose rate, a relatively small air gap is critical for Bragg peak treatment planning.

FIG. 6. Upper view: the single spot (1000 MU/spot) 2D dose rate distribution for 250 MeV proton beam at central axis plane evolves in water phantom with air gaps of 5, 15, and 25 cm, respectively; lower view: the spot dose rate at the central axis (the three sections represent the 20 cm transport in water, air gaps, and the residual range in water).

Disclosed embodiments comprise targeting the ionizing radiation. In embodiments, the ionizing radiation is targeted to a point or area within the target tissue plus an expansion margin, for example between the center of the target tissue and a point within 5 cm beyond (extending outwardly from the center) the edge of the target tissue. For example, in embodiments, the ionizing radiation is targeted so that the Bragg peak of the ionizing radiation coincides with a point within or near the perimeter or margin of the target tissue, such as a distal (with respect to the ionizing radiation transport direction) edge, a proximal edge, or a lateral edge of the target tissue.

For example, in embodiments the ionizing radiation is targeted to a distance from perimeter/margin of the target tissue, such as an edge of a tumor. For example, in embodiments, the ionizing radiation is targeted 1 mm from an edge of the target tissue, or 2 mm from an edge of the target tissue, or 3 mm from an edge of the target tissue, or 4 mm from an edge of the target tissue, or 5 mm from an edge of the target tissue, or 6 mm from an edge of the target tissue, or 7 mm from an edge of the target tissue, or 8 mm from an edge of the target tissue, or 9 mm from an edge of the target tissue, or 10 mm from an edge of the target tissue, or 11 mm from an edge of the target tissue, or 12 mm from an edge of the target tissue, or 13 mm from an edge of the target tissue, or 14 mm from an edge of the target tissue, or 15 mm from an edge of the target tissue, or 16 mm from an edge of the target tissue, or 17 mm from an edge of the target tissue, or 18 mm from an edge of the target tissue, or 19 mm of an edge of the target tissue, or 20 mm of an edge of the target tissue, or more.

For example, in embodiments the ionizing radiation is targeted to or around the perimeter/margin of the target tissue, such as an edge of a tumor. For example, in embodiments, the ionizing radiation is targeted to within 1 mm of an edge of the target tissue, or within 2 mm of an edge of the target tissue, or within 3 mm of an edge of the target tissue, or within 4 mm of an edge of the target tissue, or within 5 mm of an edge of the target tissue, or within 6 mm of an edge of the target tissue, or within 7 mm of an edge of the target tissue, or within 8 mm of an edge of the target tissue, or within 9 mm of an edge of the target tissue, or within 10 mm of an edge of the target tissue, or within 11 mm of an edge of the target tissue, or within 12 mm of an edge of the target tissue, or within 13 mm of an edge of the target tissue, or within 14 mm of an edge of the target tissue, or within 15 mm of an edge of the target tissue, or within 16 mm of an edge of the target tissue, or within 17 mm of an edge of the target tissue, or within 18 mm of an edge of the target tissue, or within 19 mm of an edge of the target tissue, or within 20 mm of an edge of the target tissue, or more.

In embodiments, the ionizing radiation is targeted toward the center of a tumor, or within a distance on either side from the center. For example, in embodiments, the ionizing radiation is targeted to within 1 mm of the center of the target tissue, or within 2 mm of the center of the target tissue, or within 3 mm of the center of the target tissue, or within 4 mm of the center of the target tissue, or within 5 mm of the center of the target tissue, or within 6 mm of the center of the target tissue, or within 7 mm of the center of the target tissue, or within 8 mm of the center of the target tissue, or within 9 mm of the center of the target tissue, or within 10 mm of the center of the target tissue, or within 11 mm of the center of the target tissue, or within 12 mm of the center of the target tissue, or within 13 mm of the center of the target tissue, or within 14 mm of the center of the target tissue, or within 15 mm of the center of the target tissue, or within 16 mm of the center of the target tissue, or within 17 mm of the center of the target tissue, or within 18 mm of the center of the target tissue, or within 19 mm of the center of the target tissue, or within 20 mm of the center of the target tissue, or more.

In further embodiments, the ionizing radiation is targeted to a point between an edge of the target tissue and the three-dimensional center of the target tissue.

Disclosed embodiments can produce field dose rates of, for example, at least 40 Gy/s, or more. For example, in embodiments, the dose rate is at least 40 Gy/s, at least 42 Gy/s, at least 44 Gy/s, at least 46 Gy/s, at least 48 Gy/s, at least 50 Gy/s, at least 52 Gy/s, at least 56 Gy/s, at least 58 Gy/s, at least 60 Gy/s, at least 62 Gy/s, at least 64 Gy/s, at least 66 Gy/s, at least 68 Gy/s, at least 70 Gy/s, at least 72 Gy/s, at least 74 Gy/s, at least 76 Gy/s, at least 78 Gy/s, at least 80 Gy/s, at least 82 Gy/s, at least 84 Gy/s, at least 86 Gy/s, at least 88 Gy/s, at least 90 Gy/s, at least 92 Gy/s, at least 94 Gy/s, at least 96 Gy/s, at least 98 Gy/s, at least 100 Gy/s, at least 102 Gy/s, at least 104 Gy/s, at least 106 Gy/s, at least 108 Gy/s, at least 110 Gy/s, at least 112 Gy/s, at least 114 Gy/s, at least 116 Gy/s, at least 118 Gy/s, at least 120 Gy/s, at least 122 Gy/s, at least 124 Gy/s, at least 126 Gy/s, at least 128 Gy/s, at least 130 Gy/s, at least 132 Gy/s, or more.

Disclosed embodiments further comprise computer-readable instructions for determining the employment of the range shifters and compensators. For example, in disclosed embodiments, computer-readable instructions can comprise instructions for calculating the number, thickness, and placement of URS and RCs based upon the characteristics of the non-shifted and non-compensated ionizing radiation, the target treatment depth, and the target's three-dimensional shape.

Methods of Use

Methods disclosed herein can comprise producing shifted and compensated ionizing radiation from an initial ionizing radiation source. For example, in embodiments, the initial ionizing radiation source can comprise protons, α-rays, carbon ions, other ion rays, and combinations thereof. In embodiments, methods comprise the use of a cyclotron or a synchrotron to produce an ionizing radiation, which is then subject to range shifter and range compensation.

In embodiments, range shifting comprises the use of multiple plastic plates, for example transparent amorphous thermoplastic plates such as polycarbonate plastic plates, with varying thicknesses, to reduce the range of the initial ionizing radiation to the desired range. For example, in embodiments, an inverse-planning tool is used to determine the number, position, and thickness of the individual URS plates necessary to lower the range of the initial ionizing radiation to the desired range.

In embodiments, range compensation comprises the use of at least one amorphous thermoplastic contour such as a polycarbonate plastic contour, to adjust the ranges of the initial ionizing radiation to the desired depths. For example, in embodiments, an inverse-planning tool is used to determine the contour thicknesses of an RC plate necessary to adjust the ranges of the initial ionizing radiation to the desired depths to adapt the target distal edge. In embodiments, the range shifted and compensated ionizing radiation field can be further used to treat a target tissue, for example a cancerous tissue. For example, disclosed methods of use can comprise;
a. diagnosing a cancerous tissue;
b. mapping the cancerous tissue;
c. developing a radiotherapy treatment plan to administer an effective amount of ionizing radiation to the cancerous tissue; and
d. administering the range shifted and compensated ionizing radiation to the cancerous tissue.

In embodiments, disclosed methods can comprise diagnosis of a cancerous tissue, for example by the use of lab tests, imaging tests, biopsy, and the like. For example, elevated or depressed levels of certain substances in the body can be a sign of cancer. Therefore, lab tests of blood, urine, or other body fluids or cells can measure these substances and help doctors make a diagnosis. In embodiments, lab tests involve testing blood or tissue samples for tumor markers. Tumor markers are substances that are produced by cancer cells or by other cells of the body in response to cancer. Most tumor markers are made by normal cells and cancer cells, but they are generally produced at much higher levels by cancer cells.

Disclosed methods can further comprise the use of imaging tests to identify cancerous tissue. For example, imaging tests visualize areas inside the body that help identify cancerous tissue. Disclosed methods comprising imaging tests can comprise, for example, CT scans, wherein an x-ray machine linked to a computer takes a series of pictures of internal organs from different angles. These pictures are used to create detailed 3-D images of the inside of the body.

Further methods comprise use of magnetic resonance imaging (MRI). An MRI uses a powerful magnet and radio waves to take pictures of the body in sections, which can show the difference between healthy and unhealthy tissue.

Further disclosed methods can comprise nuclear scanning, which uses uses radioactive material to image the inside of the body. This type of scan may also be called radionuclide scan.

Additional disclosed methods of treatment can comprise bone scanning, which is a type of nuclear scan that is used to identify anomalies in bone.

Further disclosed methods can comprise a positron emission tomography (PET) scan. A PET scan is an imaging test that allows your doctor to check for diseases in your body. The scan uses a special dye containing radioactive tracers. These tracers are either swallowed, inhaled, or injected into a vein in a patient's arm depending on what part of the body is being examined.

Further diagnostic methods suitable for use in disclosed embodiments can comprise ultrasound. An ultrasound exam uses high-energy sound waves that “echo” off tissues inside the body. A computer uses these echoes to create pictures of areas inside your body.

In embodiments, multiple diagnostic methods can be used to identify prospective areas of treatment. In embodiments, the area of treatment comprises a tumor, for example malignant tumors.

Once diagnosed, the area of treatment can be mapped and a treatment plan developed. For example, after a target tissue is three-dimensionally mapped, appropriate URS, RC, dosages, and beam angles are determined.

The treatment plan can then be applied as administration of range shifted and compensated ionizing radiation at an appropriate dose. For example, disclosed methods can comprise treatment dose of, for example, at least 1.8 Gy/fraction, or more. For example, in embodiments, the dose is at least 1.8 Gy/fraction, at least 2 Gy/fraction, at least 3 Gy/fraction, at least 4 Gy/fraction, at least 5 Gy/fraction, at least 6 Gy/fraction, at least 8 Gy/fraction, at least 10 Gy/fraction, at least 12 Gy/fraction, at least 14 Gy/fraction, at least 16 Gy/fraction, at least 18 Gy/fraction, at least 20 Gy/fraction, at least 22 Gy/fraction, at least 24 Gy/fraction, at least 26 Gy/fraction, at least 28 Gy/fraction, at least 30 Gy/fraction, at least 32 Gy/fraction, at least 34 Gy/fraction, at least 36 Gy/fraction, at least 38 Gy/fraction, at least 40 Gy/fraction, at least 42 Gy/fraction, at least 44 Gy/fraction, at least 46 Gy/fraction, at least 48 Gy/fraction, at least 50 Gy/fraction, at least 52 Gy/fraction, at least 54 Gy/fraction, at least 56 Gy/fraction, at least 58 Gy/fraction, at least 60 Gy/fraction, at least 62 Gy/fraction, at least 64 Gy/fraction, at least 66 Gy/fraction, at least 68 Gy/fraction, at least 70 Gy/fraction, or more.

In embodiments, the fraction dose is, for example, 1.8 Gy/fraction, or more. For example, in embodiments, the dose is 1.8 Gy/fraction, 2 Gy/fraction, 3 Gy/fraction, 4 Gy/fraction, 5 Gy/fraction, 6 Gy/fraction, 8 Gy/fraction, 10 Gy/fraction, 12 Gy/fraction, 14 Gy/fraction, 16 Gy/fraction, 18 Gy/fraction, 20 Gy/fraction, 22 Gy/fraction, 24 Gy/fraction, 26 Gy/fraction, 28 Gy/fraction, 30 Gy/fraction, 32 Gy/fraction, 34 Gy/fraction, 36 Gy/fraction, 38 Gy/fraction, 40 Gy/fraction, 42 Gy/fraction, 44 Gy/fraction, 46 Gy/fraction, 48 Gy/fraction, 50 Gy/fraction, 52 Gy/fraction, 54 Gy/fraction, 56 Gy/fraction, 58 Gy/fraction, 60 Gy/fraction, 62 Gy/fraction, 64 Gy/fraction, 66 Gy/fraction, 68 Gy/fraction, 70 Gy/fraction, or the like.

In embodiments, the fraction dose is not more than 1.8 Gy/fraction, not more than 2 Gy/fraction, not more than 3 Gy/fraction, not more than 4 Gy/fraction, not more than 5 Gy/fraction, not more than 6 Gy/fraction, not more than 8 Gy/fraction, not more than 10 Gy/fraction, not more than 12 Gy/fraction, not more than 14 Gy/fraction, not more than 16 Gy/fraction, not more than 18 Gy/fraction, not more than 20 Gy/fraction, not more than 22 Gy/fraction, not more than 24 Gy/fraction, not more than 26 Gy/fraction, not more than 28 Gy/fraction, not more than 30 Gy/fraction, not more than 32 Gy/fraction, not more than 34 Gy/fraction, not more than 36 Gy/fraction, not more than 38 Gy/fraction, not more than 40 Gy/fraction, not more than 42 Gy/fraction, not more than 44 Gy/fraction, not more than 46 Gy/fraction, not more than 48 Gy/fraction, not more than 50 Gy/fraction, not more than 52 Gy/fraction, not more than 54 Gy/fraction, not more than 56 Gy/fraction, not more than 58 Gy/fraction, not more than 60 Gy/fraction, not more than 62 Gy/fraction, not more than 64 Gy/fraction, not more than 66 Gy/fraction, not more than 68 Gy/fraction, not more than 70 Gy/fraction, or the like.

In embodiments, the range shifted and compensated ionizing radiation is administered in multiple fields. For example, in embodiments, 2 fields are administered, or 3, or 4, or 5, or 6, or 7, or 8, or 9, or the like. In embodiments, at least 2 fields are administered, or at least 3, or at least 4, or at least 5, or at least 6, or at least 7, or at least 8, or at least 9, or the like. In embodiments, not more than 2 fields are administered, or not more than 3, or not more than 4, or not more than 5, or not more than 6, or not more than 7, or not more than 8, not more than 9, not more than 10, not more than 11, not more than 12, not more than 13, not more than 14, not more than 15, or the like.

In embodiments, the angles at which the fields of range shifted and compensated ionizing radiation are administered to the target tissue are equivalent between the fields. For example, in embodiments wherein 5 fields are administered, the angles at which the fields are administered can be 72 degrees apart. The angle between the fields can be determined as appropriate to meet the clinical requirement.

The frequency of employment of the disclosed methods can be determined based on the nature and location of the particular area being treated. In certain cases, however, repeated treatment in the future may be desired to achieve optimal results.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of Lung Tumors

Using the highest single-energy beam of the cyclotron or synchrotron machine, the Bragg peak plan achieved sufficient beam current for FLASH dose rate; meanwhile, multiple-field inverse optimization treatment planning with range pulling-back devices (URS) in the beam path enabled the treatment planning to eliminate the exit dose beyond targets. An inverse algorithm was developed based on matRad platform [Wieser H P, Cisternas E, Wahl N, et al. Development of the opensource dose calculation and optimization toolkit matRad. Med Phys 2017; 44:2556-2568] to achieve a uniform dose distribution by MFO; once the treatment plan meets the target coverage and OARs constraints, a raytracing method created a range compensator for each field. As shown in FIG. 1(e), a 5-field IMPT plan used universal range shifters (URS) and range compensators (RC) for lung cancer FLASH-RT, and the proton range was tailored to adapt to the target distal edge.

The 3D dose rate was quantified by the dose averaged dose rate (DADR) [Water S, Safai S, Schippers J M, et al. Towards FLASH proton therapy: the impact of treatment planning and machine characteristics on achievable dose rates. Acta Oncologica 2019; 58:10, 1463-1469. https://doi:10.1080/0284186X.2019.1627416]. It will be appreciated that other approaches may also be employed. PBS spot center had a maximum dose rate, and the dose rate dropped radially from the center to the lateral of a spot. Here we adopted the spot peak dose rate (SPDR) definition, firstly proposed by van Marlen et al [van Marlen P, Dahele M, Folkerts M, et al. Bringing FLASH to the Clinic: Treatment Planning Considerations for Ultrahigh Dose-Rate Proton Beams, Int J Radiation Oncol Biol Phys 2020; 106(3): 621-629. https://doi.org/10.1016/j.ijrobp.2019.11.011] for FLASH planning study to define the central axis's maximum dose rate. An in-house 3D PBS dose rate calculation tool was developed in MATLAB using pencil beam convolution superposition algorithm with heterogeneity correction, Gaussian spot profile (σ=3.5 mm), and spot delivery time structures modeled, to calculate the dose rate. A dose rate volume histogram (DRVH) method was also used to quantify the dose coverage of the target and OARs. At the same time, to quantitatively assess FLASH dose rate, a dose rate coverage index V_{40Gy/s} was defined that represented the percentage of the volume receiving dose rate ≥40 Gy/s for target and OARs.

FIG. 1. Schematic diagram of non-transmission FLASH IMPT planning used universal range shifters (URS) and range compensators (RC). The URS and RC were placed in the beam path for illustration purpose only. (a) A phantom example that used a 6-mm distal margin to determine the location of the proton stoppage. The dots represent where the protons stop, and the integrated water equivalent thickness (WET) distance from the body contour was calculated to determine the range pulling-back and compensator contour; (b) dose distribution using a single-energy layer based on the spot map of (a); (c) beam-eye-view of 2D range compensation calculated by a ray-tracing method; (d) the 1D range compensation at the central axis; (e) a 5-field beam arrangement for a lung treatment plan, with the upper and lower corner illustrating one of the 5-RC with two different views.

2.1 Inverse Planning to Achieve IMPT Treatment Plans

2.1.1 Ray Tracing to Calculate the Range Compensation

A single-energy beam was customized to generate the intensity-modulated spot map via the inverse planning platform. As shown in FIG. 1(a), a uniform margin of 6-mm on the CTV was used to contain the spot distribution in-depth direction. The 90% of dose falloff was used as the proton range for spot map generation. The water equivalent thickness (WET) of each pencil beam proton radiographic track (denoted by WETi (x, y, z)) was calculated by Eq.1, and rsp(x, y, z) represents the relative stopping power (rsp) of each voxel of the 3D CT images. The integral step in Eq.1 was accurately computed with a raytracing algorithm. Each pencil beam range pulling-back is calculated by Rᵢ, where R_{Eo}, is the range of the highest energy in water. All FLASH-RT Bragg peak plans used a 5-field beam arrangement, and an MFO method was used to generate spot maps.

Equation 1

WETᵢ(x,y,x)=∫₀^{depth}rsp(x,y,z)dlᵢ

Rᵢ=R_{E0}=WETᵢ(x,y,z) (1)

2.1.2 Universal Range Shifter (URS) and Range Compensator (RC)

The total range compensation for each field was achieved by using a URS and an RC. The thickness of a URS changes from 0 to 34 cm, with the assistance of RC, enables the treatment of tumors at all depths.

The URS consisted of 6 polycarbonate plastic plates of thicknesses of 1, 2, 3, 7, 7, and 14 cm WET, generating 35 discrete range pulling-backs with a depth resolution of 1 cm. Each range shifter plate was driven by a standalone step motor to move “in” and “out” of the beam path, and the “in” and “out” combination of the six plates is similar to a binary system that can generate the correct range pulling back. The range plate combinations for 35 discrete range pulling-backs are depicted in Table 2.

FIG. 1(e) shows the schematic of the URS system, and the thicker range shifters are placed closer downstream. The thinner range shifters were more upstream, a design consideration to minimize the transport distance of scattered proton beams to reduce spot size and preserve a high SPDR. The desired proton ranges were achieved by moving the range shifter plates “in” and “out” of the beam path. The thickness of URS used in each beam path was be calculated using R_{URS} in Eq.2. The max thickness of Rc is determined by R_{C} in Eq. 2. Therefore, the total range pulling-back capacity is between 0 and R_{E0} cm that can accommodate the deepest targets to the superficial targets. The range compensation Ri of each proton trace under each field is calculated and stored by a 3D data matrix. The data sets can be used to construct 3D printed compensators conveniently. As shown in FIG. 1(e), the RC is presented on the right upper and lower corner.

Equation 2

f(x,y,z)=max(WETᵢ(x,y,z))−min(WETᵢ(x,y,z))

R_{C}=dcm and R_{URS}=(R_{E0}−f)cm (2)

Similar to the compensator design for scattering proton systems, a smearing method [Moyers M F, Miller D W, Bush D A, Slater J D. Methodologies and tools for proton beam design for lung tumors. Int J Radiat Oncol Biol Phys 2001; 49(5):1429-38.] was used to design the RC to manage range uncertainties. A robust plan is important to achieve the clinical intention for the target and OARs. The major uncertainties resulting from setup and motion can cause large range uncertainties. A 3.5% CT Hounsfield unit (HU) to relative stopping conversion uncertainties and 3-mm setup errors were used when applying the smearing method.

2.2 Spot Map Optimization to Improve Plan Quality and Dose Rate

Next, the minimal MU/spot or minimal treatment room beam current in nanoampere (nA) determined the dose rate of each energy layer, and the minimal MU/spot and dose rate needs to be further optimized to reach the FLASH dose rate threshold. An in-house algorithm was developed to generate an optimal spot map via two steps: first, an initial minimal MU/spot threshold (w₀) was used for inverse optimization and a dense spot map with a defined spot spacing was generated; second, the low weighting spots were merged to new spots by applying both a distance threshold rₜ and a weighting factor of wₜ, in which the rₜ is a ratio of spot spacing. The wₜ is a factor-based minimal MU/spot requirement for FLASH dose rate. As shown in Eq.3, the weights are combined as wₘ, and the spot coordinates are calculated based on their original coordinates and weighting fractions using Eq.4. The final spot location is determined by applying the coordinate threshold rₜ, described using Eq. 5. In general, there are two considerations for applying the second step to generate the final spot maps. First, as the minimal MU/spot determines the SPDR of the layer, by merging the low weighting spots, a high SPDR can be achieved. Second, the low weighted spots significantly contribute to maintaining a good plan quality as with conventional IMPT plan optimization. By merging lower weighted spots to the nearby ones, the spot distribution pattern is minimally changed, but better plan dosimetry distribution is achievable.

Continuous optimization was performed to fine-tune the spot weights to further improve target uniformity and OARs sparing. By iteratively applying the second step of spot map optimization, the dose rate was continuously improved. The efficacy of the spot map optimization was tested using a C-shape target that surrounds a central avoidance core structure. As shown in FIG. 2, (a) is the spot map of one field using an initial 400 MU/spot threshold, (d) is the spot map after applying the spot map optimization process, (b) and (e) are the 2D dose distribution comparison for a selected slice, (c) and (f) are the DVH and DRVH comparison. It was evident that the low dose region was reduced, and the conformity was improved, as can be seen from the 2D dose distribution, and the DVH of the core structure resultingly shifted towards the lower dose end substantially. The dose rates to body, target, and core structure were increased, illustrated in (f).

$${w}_{m} = {w}_{i} + {w}_{i + 1} \qquad ( 3 )$$

$$\vec{{r}_{m} ( x , y )} = \frac{{w}_{i}}{{w}_{m}} \vec{{r}_{i} ( x , y )} + \frac{{w}_{i + 1}}{{w}_{m}} \vec{{r}_{i + 1} ( x , y )} \qquad ( 4 )$$

$$\vec{{r}_{m} ( x , y )} = arg min \vert \vec{{r}_{m} ( x , y )} - \vec{{r}_{t} ( x , y )} \vert \qquad ( 5 )$$

FIG. 2 shows an example of spot distribution and weight optimization that can effectively improve the plan quality and FLASH-RT dose rate distribution. (a) and (d) the spot maps before and after the spot map optimization process; (b) and (e) the 2D dose distribution comparison; (c) and (f) the DVH and DRVH comparisons before and after spot map optimization. A dashed line from the DRVH marks the 40 Gy/s threshold.

2.3 Spot Dose Rate Evolution in Range Shifter, Air, and Phantom

The MCS between the protons and USR and RC can enlarge the spot divergence significantly. Equivalently, the scattering effects have also resulted in progressive shortening of the effective-SSD of the beam. At a shorter effective-SSD, proton fluence decreases more quickly due to a larger inverse square effect, and the spot size increases more rapidly. FIG. 6 illustrates the dose rate distribution for a 250 MeV single spot with 1000 MU in a water phantom. We mimicked 5, 15, and 25 cm air gaps between the RC and the phantom surface to calculate the spot dose rate at the central axis in water changing with air gaps. It was clear that spot dose rate decreased when the air gap increased, and the central axis dose rate at the Bragg peak is reduced by a factor of ˜2 between 5 cm and 25 cm air gaps. During FLASH plan optimization, minimizing the air gap plays an important role in maintaining proton fluence intensity and a smaller penumbra, which is crucial for the OAR sparing. Meanwhile, a large spot size caused by MCS and a large air gap will significantly reduce the spot dose rate and the treatment field's mean dose rate. To achieve a higher spot dose rate, a relatively small air gap is critical for Bragg peak treatment planning.

FIG. 6. Upper view: the single spot (1000 MU/spot) 2D dose rate distribution for 250 MeV proton beam at central axis plane evolves in water phantom with air gaps of 5, 15, and 25 cm, respectively; lower view: the spot dose rate at the central axis (the three sections represent the 20 cm transport in water, air gaps, and the residual range in water).

3.3 Feasibility Study Using Both Phantom and Patient Images

A rectangular water “phantom” with a C-shape target was used for planning to investigate the dosimetry quality and dose rate distribution for both transmission and Bragg peak plans. At the same time, six consecutive lung cancer patients previously treated with proton SBRT at our facility were re-planned to receive both transmission and Bragg peak FLASH plans to assess the dose and dose rate distribution. The planning goal of using Bragg peak FLASH-RT was to achieve a comparable V_{40Gy/s} dose rate coverage for critical OARs but substantially improved OAR sparing while using OAR dose constraints from the original clinical treatment parameters. Marlen et al. reported a FLASH transmission study using 8 to 12 non-coplanar beams for lung cancer planning. The reason to use more fields was to reduce normal lung dose and improve the target conformality. To make a “fair” dosimetry comparison, this study used 5-field plans with even angle separation (72 degrees) for both transmission and Bragg peak plans. The phantom plan used a prescription of 50 Gy in 1 fraction, and the patient plans used a standard-of-care prescription of 34 Gy in 1 fraction. The minimal MU to maintain the FLASH dose rate for the transmission and Bragg peak plans was 400 MU/spot and 1200 MU/spot, respectively. The 3D dose rate quantification used the DADR method, and DRVHs are investigated for both phantom and patient plans.

3. Results

3.1 Bragg Peak FLASH-RT Planning Using Phantom

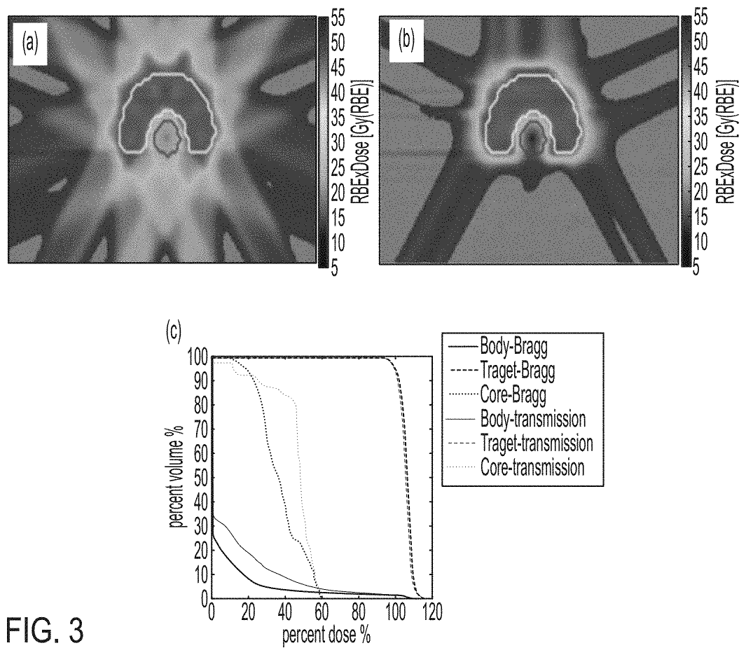

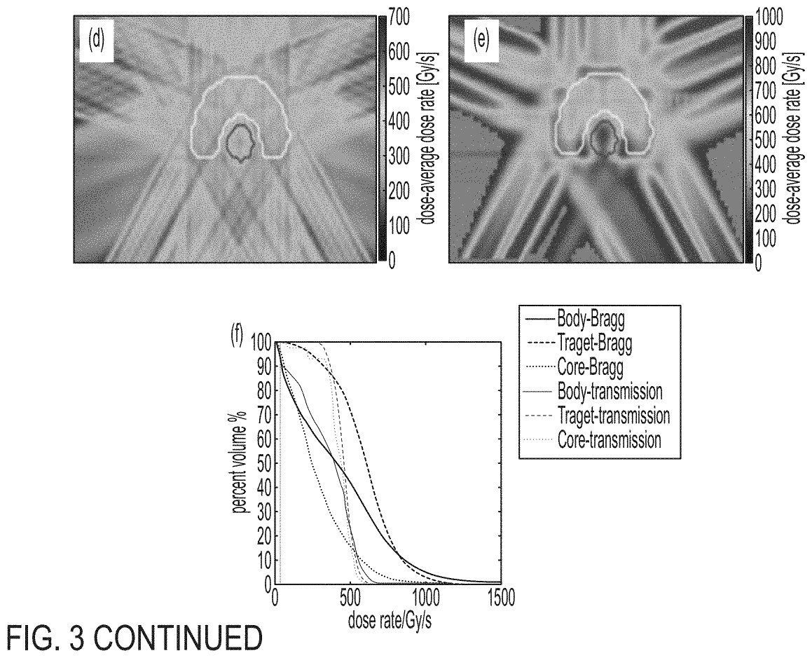

The phantom planning used five fields with 72 degrees equal angle separation, and the distance between the core and C-shape target was about 1.5 cm. FIGS. 3 (a) and (b) are the 2D dose distribution for a selected slice. The Bragg peak plan resulted in less low dose scattering cloud and integral dose than the transmission plans due to the non-existence of exit dose with the Bragg peak method. The target coverage and uniformity were nearly identical but with much less dose spillage to the body and core structures. As shown in FIG. 3(c), the DVHs of the body and core had a large separation between the two FLASH delivery methods, demonstrating that the Bragg peak plans reduce dose spillage from low to a medium level significantly. FIGS. 3(d) and (e) are the DADR dose rate distribution for the same image slice, and FIG. 3(f) is the DRVH comparison. It was evident that the transmission plans tended to generate a higher dose rate distribution versus Bragg peak plans. However, Bragg peak can also reach FLASH-RT threshold 40_{Gy/s} by increasing the minimal MU/spot (1200 MU/spot) and via dose rate optimization. As illustrated in FIG. 3(f), after applying the spot map optimization, the V_{40Gy/s} coverage of the body and surrogate OAR core structure was as high as 95%. The phantom dosimetry and dose rate comparison indicated that the Bragg peak plans can achieve similar target coverage, but much better OAR sparing compared to the transmission plans. At the same time, the FLASH-RT dose rate can be successfully maintained by the Bragg peak plan.

FIG. 3. Transmission ((a) & (d)) vs. Bragg peak ((b) & (e)) planning using 250 MeV proton beams for C-shape target in a water phantom: (a) and (b) are the 2D dose distribution for a selected slice, (c) is the DVH comparison between them; (d) and (e) are the 2D dose rate distribution, and (f) is the DRVH comparison. A dashed line from the DRVH marks the 40_{Gy/s} threshold.

3.2 Lung Hypofractionation Plan Study

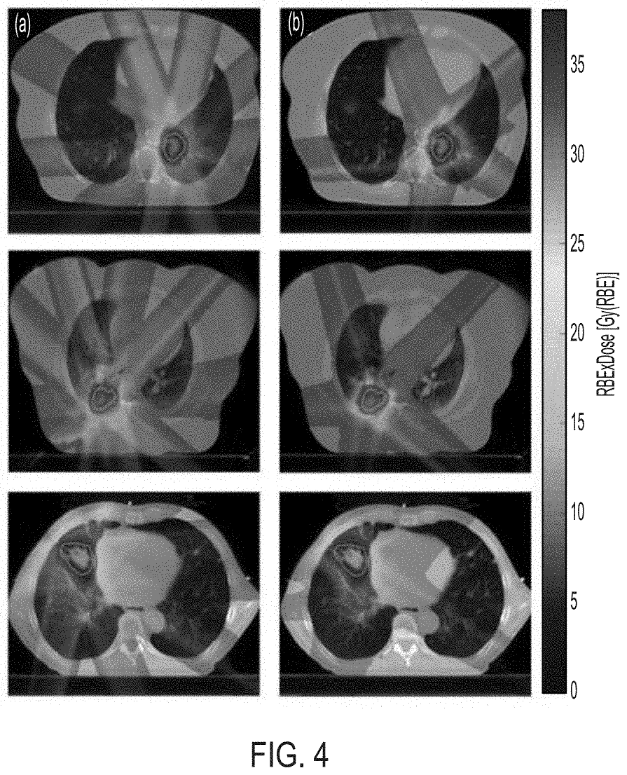

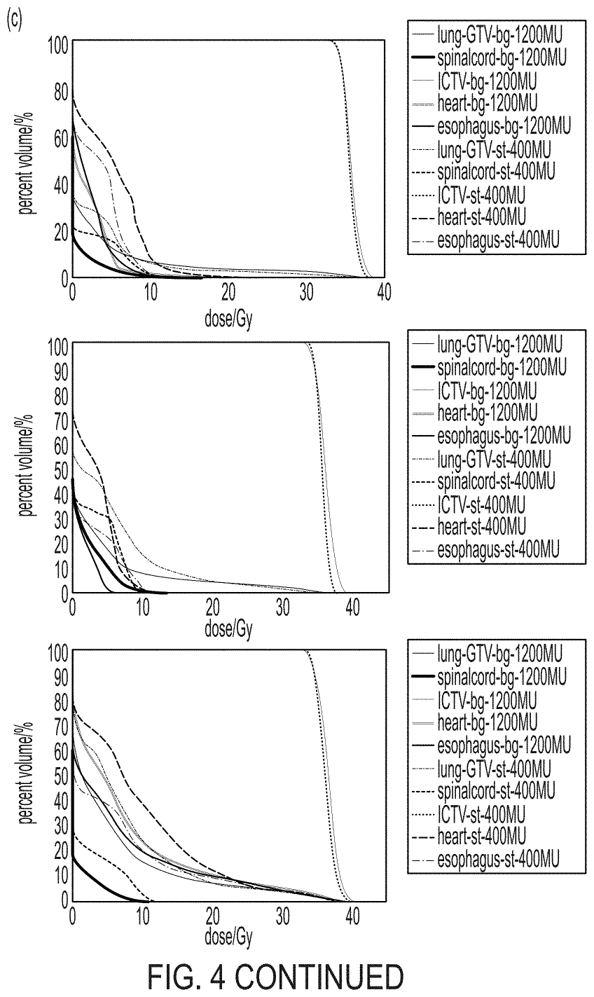

The 2D dose distribution is displayed in FIGS. 4 (a) and (b) for the three selected cases. As illustrated from the dose color wash, the Bragg peak plans were superior in low and medium dose regions. The scattering dose cloud and integral dose were significantly less in dose distributions of Bragg peak plans for all three cases. The 3-DVH (FIG. 4(c)) also showed a significant dose-volume reduction for lung-GTV, esophagus, spinal cord, and heart when using the Bragg peak method for FLASH planning. Table 1 compiles the dosimetry parameters based on the RTOG0915 protocol for both transmission and Bragg peak IMPT plans [RTOG0915. https://www.nrgoncology.org/Clinical-Trials/Protocol/rtog-0915?filter=rtog-0915] for all six lung patients. D2% represents the high dose region and dose uniformity of the target. The Bragg peak plans yielded slightly worse uniformity than the transmission plan (110.6% vs. 112.2% of the prescription dose) due to the increase of minimum MU/spot. Most of the OAR dose metrics, however, are superior for Bragg peak plans. For instance, for lung-GTV, the mean volume of V7Gy is reduced from 724.9 cc to 492.6 cc, which corresponds to a volume reduction up to 32% (p=0.001); the mean volume of V7.4Gy is reduced from 672.8 cc to 468.7 cc with an irradiated volume reduction of 30% (p=0.002). Additionally, while the doses to the esophagus, heart, and spinal cord are sensitive to the target locations and beam arrangements, some of those OARs could be completely spared in Bragg peak plans if there were no beam passing through or toward them. Given the larger statistical errors and the heterogeneity of tumor sites in this initial cohort, these differences were not statistically significant (p>0.05).

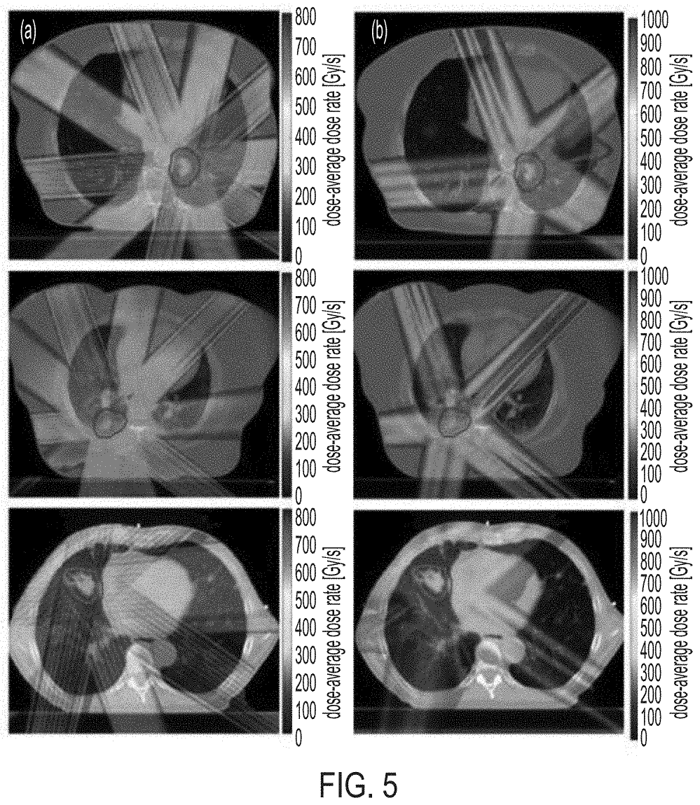

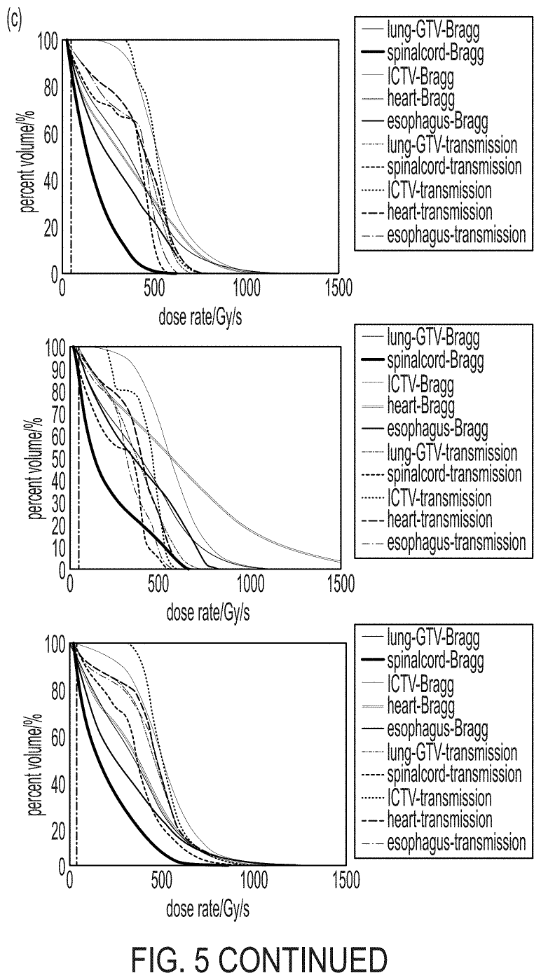

FIG. 5 is the 2D dose rate distribution and DRVH comparison for the three selected lung cancer patients. The left column shows the dose rate distribution of the transmission plan, illustrating that the proton dose rate attenuates with the depth when passing through tissues, and the exit dose rate is much lower than the entrance dose rate for each of the single fields. The middle column is the dose rate distribution for Bragg peak plans with high dose rate strips and low dose rate valleys observed in each of the fields. As is known, the freedom of plan optimization includes spot maps, spot weights, number of spots and fields, and minimal MU/spot, etc. Using 400 MU/spot and 1200 MU/spot for transmission and Bragg peak plans, the increase of minimum MU/spot will increase the difficulty to achieve good uniformity and OARs sparing for the Bragg peak plan. All parameters are optimized except for energy and the number of fields to fulfill the required dosimetry objects via inverse optimization. Here the highly modulated fluence serves as compensation for reducing flexibility with increasing MU/spot in maintaining a higher 3D dose rate. The inverse algorithm plays a crucial role in achieving a uniform dose distribution via non-uniform dose fluence by the MFO method. The V_{40Gy/s} dose rate coverage was compiled in Table 1. All targets can reach almost 100% V_{40Gy/s}, and the mean V_{40Gy/s} coverage of all OARs is at least >91.0±3.8% (spinal cord of Bragg peak plans) for both transmission and Bragg peak plans. In all, the dose distribution, DVHs, and dosimetry metrics comparison demonstrate substantially improved sparing for lung, cord, heart, and esophagus with Bragg peak plans; in contrast, those plans preserved the FLASH dose rate.

Table 1. Dosimetry and dose rate coverage of V_{40Gy/s} comparison for transmission and Bragg peak IMPT plans for all six lung cases. The dosimetry comparison used RTOG 0915 metrics. Both dose and dose rate statistics used the averaged values for all six cases. The last row of the table represents the averaged V_{40Gy/s} for both target and OARs.

FIG. 4. The dose comparisons between transmission and Bragg peak plans for three selected lung patients using the same beam arrangement. The right and middle columns represent transmission and Bragg peak plans, respectively. (a) and (b) 2D dose distribution for selected slices, (c) DVH comparison between the two types of plans.

FIG. 5. Dose rate comparisons between transmission (the left side images) and Bragg peak (the middle images) plans using the same beam arrangement. (a) and (b) 2D dose rate distribution for selected slices, (c) DRVH comparison between the two types of plans. A dashed line marks the 40_{Gy/s} threshold.

4. Discussion

While the MU definition varied between different proton vendors, the universal quantification for dose rate is to use the beam current at the treatment room. However, in treatment planning, MU is used as a basic unit to calculate the planned dose. To describe the dose rate precisely, the correlation between beam current and the number of protons per MU needs to be determined, e.g., by Monte Carlo simulation or experimental methods. The treatment planning system (TPS) capability to optimize the spot weight may vary between different TPSs. Different optimizers and dose calculation engines can result in varying plan qualities and dose rate distributions. Therefore, treatment planning strategies, the DVH, and DRVH distribution need to be extensively studied as these commercial TPSs are available for clinical application. Due to the intrinsic nature of dose rate attenuation when PBS spots pass through URS, RC, air gap, and patient tissue, the 3D dose rate distribution for OARs is a function of proton range pulling-back and minimal MU/spot. To treat shallow targets, a larger proton range pulling-back is needed; correspondingly, a larger minimal MU/spot is desired for plan optimization to maintain the FLASH dose rate for irradiation to OARs.

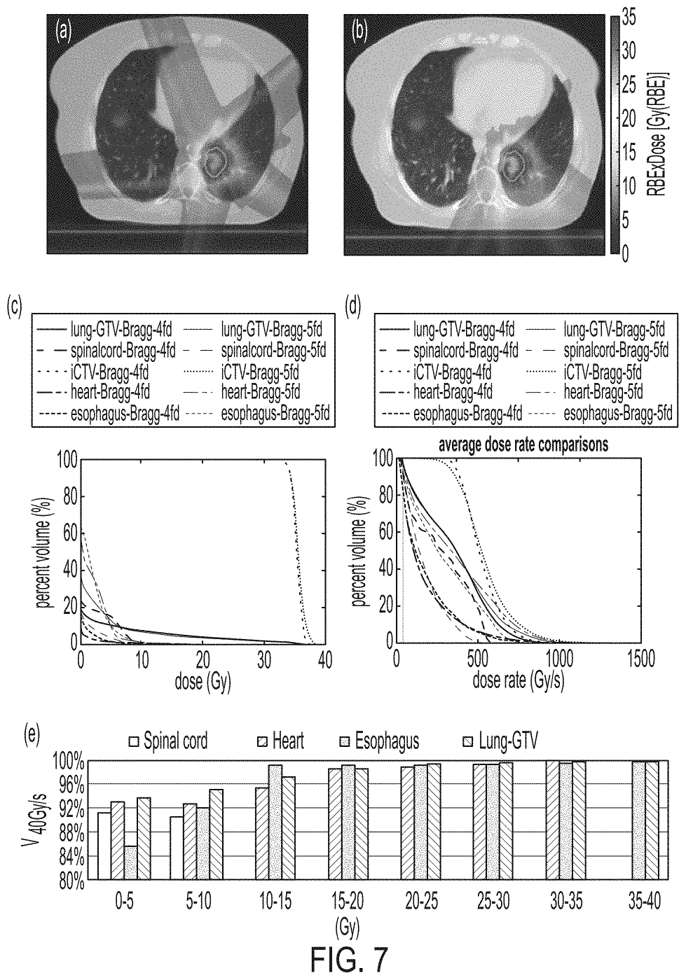

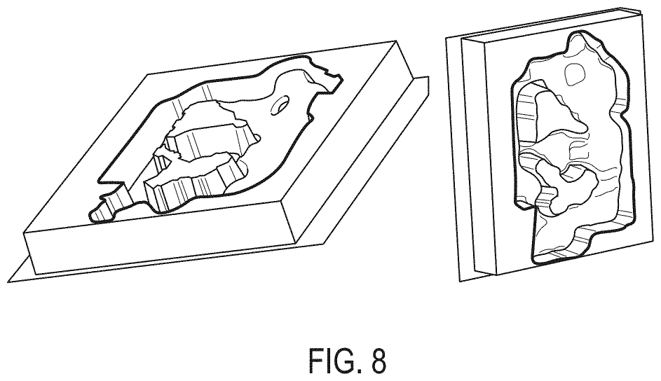

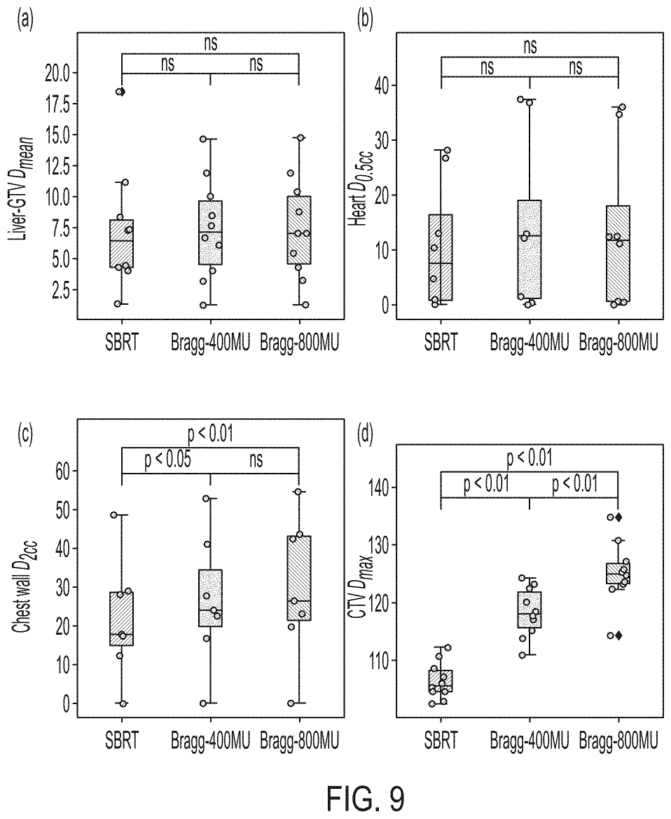

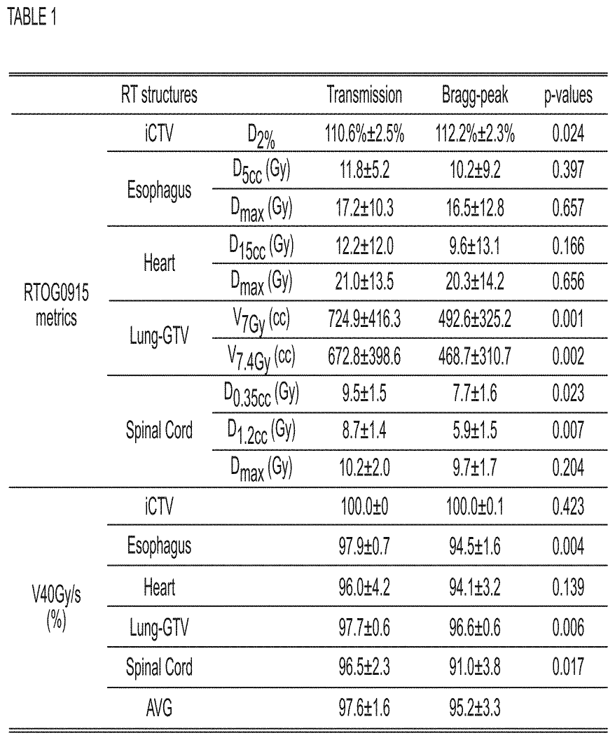

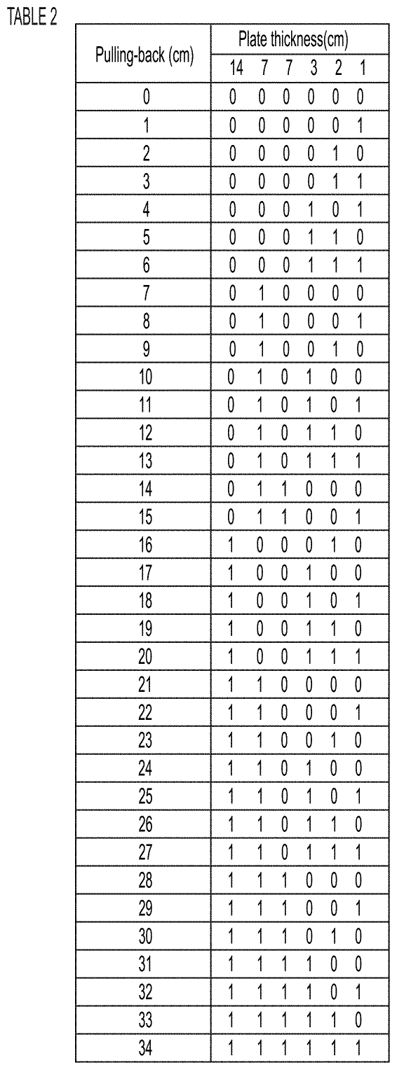

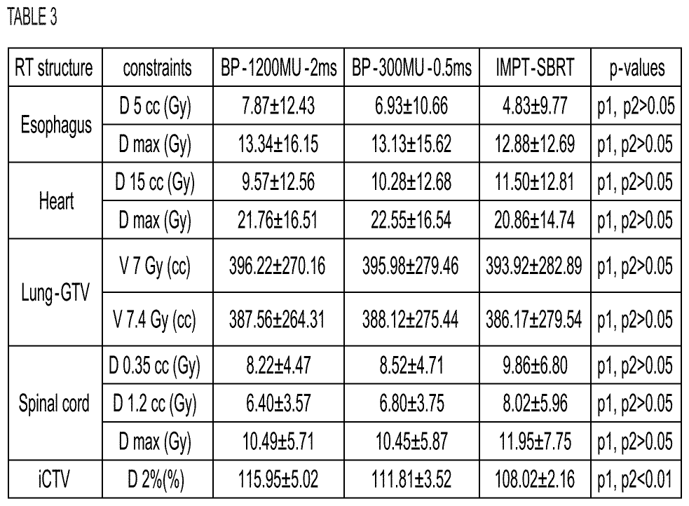

Plan optimization involving multiple parameters, including more freedom will definitely increase the flexibility and possibility for achieving better plan quality. The variable minimal MU/spot, number of beams, and beam angles are key parameters that should generally be considered during treatment planning optimization. As shown in FIG. 7, (a) uses a 4-posterior-oblique-beam arrangement which is close to conventional beam angles compared to that in (b); The contralateral lung is completely spared with almost zero doses, and dose to the esophagus and heart are also significantly reduced (DVHs from FIG. 7(c)); the V_{40Gy/s} (FIG. 7(d)) coverage for the CTV and OARs are similar. This case comparison indicates that the Bragg peak plan has much potential to further reduce OAR doses by optimizing the number of fields and beam angles. Plan robustness is extremely important to achieve the clinically intended dose to the target and OARs. A smearing method is used to consider the range uncertainties from setup and CT HU to stopping power conversion uncertainties. Motion uncertainties can also have an impact on the target treatment. As each field is delivered in under 1 second, the dose interplay effect may not be a concern for each field, while in between fields and fractions, the target motion still needs to be addressed. A 4DCT will be needed to contour moving targets considering the motion amplitude and motion region. Meanwhile, an average image may be helpful to make the treatment plan more robust. Motion mitigation strategies (DIBH, gating, etc.) may also be necessary to reduce the target margin and achieve robust treatment.

The V_{40Gy/s} coverage was slightly worse in Bragg peak plans even when the minimal MU/spot was boosted to 1200 MU/spot. We have analyzed where the low dose rate region is. We define eight dose regions with 5 Gy dose intervals from 0 to 40 Gy to quantitatively assess the dose rate statistical distribution vs. OAR doses. As shown in FIG. 7(e), there are eight dose regions from 0-5 Gy to 35-40 Gy. Most of the volume receiving FLASH dose rate coverage less than 96% had doses lower than 15 Gy. When the OARs doses are higher, the dose rate increases correspondingly. Studies indicated that the FLASH sparing effect might be correlated with the combination of dose, dose rate, and total delivery time. As studies indicated that the FLASH effect might be dose-dependent, we presume this low dose region with a slight loss of V_{40Gy/s} coverage might not be critical or of clinical significance.

FIG. 7. (a-d) An example illustrating beam angle optimization. (a) and (b) are the 2D dose distribution using different fields and field angles, (c) and (d) are the DVH, and DRVH comparison, (e) is the V_{40Gy/s} dose rate coverage vs. OAR doses from the low to high dose regions. The left side lung and most of the heart are completely spared using a beam arrangement shown by (b).

5. Conclusion

The dedicated designed URS and RS accomplished the mission of pulling proton range back from the target from distally to proximally. This novel method does not require any significant cyclotron/synchrotron or beamline updates to meet the FLASH-RT dose rate threshold using Bragg peaks. The URS is an effective tool to pull back the proton range. With the help of the RC, the proton range can be further tailored to adapt to the distal target contour to achieve a conformal dose distribution. The exit dose can be eliminated with Bragg peak FLASH plans, allowing much better OAR sparing than transmission FLASH plans. An effective inverse optimizer by taking full advantage of planning freedom, e.g., beams, angles, spot maps, spot weightings, etc., is another critical factor to achieve high-quality IMPT plans with a sufficient 3D dose rate for FLASH-RT. An efficient dose rate optimization algorithm and an accurate dose calculation engine are crucial to make Bragg peak FLASH-RT feasible in clinical practice. We have demonstrated an initial cohort planning analysis that demonstrates similar target coverage and uniformity can be maintained using Bragg peaks but with substantially improved OAR sparing relative to transmission plans. This first proof-of-concept study has demonstrated the novel method of combining range pulling back and powerful inverse optimization to achieve FLASH dose rate using currently available machine parameters.

### Example 2

### Treatment of Lung Tumor

A 26-year old male is diagnosed with a lung tumor. The cancerous tissue is imaged and mapped, and a treatment plan is devised. Using an inverse-planning optimization protocol, an optimal URS plate combination was established with the following parameters to coincide the range of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 28 | 1 | 1 | 1 | 0 | 0 | 0 |
| 26 | 1 | 1 | 0 | 1 | 1 | 0 |
| 27 | 1 | 1 | 0 | 1 | 1 | 1 |
| 29 | 1 | 1 | 1 | 0 | 0 | 1 |
| 30 | 1 | 1 | 1 | 0 | 1 | 0 |

FLASH-RT treatment is then provided to the cancerous tissue in 5 fields spaced at an angle of 72°.

### Example 3

### Treatment of Liver Tumor

A 46-year old female is diagnosed with a liver tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 18 | 1 | 0 | 0 | 1 | 0 | 1 |
| 23 | 1 | 1 | 0 | 0 | 1 | 0 |
| 19 | 1 | 0 | 0 | 1 | 1 | 0 |
| 20 | 1 | 0 | 0 | 1 | 1 | 1 |
| 21 | 1 | 1 | 0 | 0 | 0 | 0 |
| 22 | 1 | 1 | 0 | 0 | 0 | 1 |

FLASH-RT treatment is then provided to the cancerous tissue in 6 fields spaced at an angle of 60°.

### Example 4

### Treatment of Brain Tumor

A 41-year old male is diagnosed with a brain tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 21 | 1 | 1 | 0 | 0 | 0 | 0 |
| 22 | 1 | 1 | 0 | 0 | 0 | 1 |
| 23 | 1 | 1 | 0 | 0 | 1 | 0 |
| 21 | 1 | 1 | 0 | 0 | 0 | 0 |

FLASH-RT treatment is then provided to the cancerous tissue in 4 fields spaced at an angle of 90°.

### Example 5

### Treatment of Brain Tumor

A 21-year old male is diagnosed with a brain tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 26 | 1 | 1 | 0 | 1 | 1 | 0 |
| 32 | 1 | 1 | 1 | 1 | 0 | 1 |
| 27 | 1 | 1 | 0 | 1 | 1 | 1 |
| 28 | 1 | 1 | 1 | 0 | 0 | 0 |
| 33 | 1 | 1 | 1 | 1 | 1 | 0 |
| 29 | 1 | 1 | 1 | 0 | 0 | 1 |
| 30 | 1 | 1 | 1 | 0 | 1 | 0 |
| 31 | 1 | 1 | 1 | 1 | 0 | 0 |

FLASH-RT treatment is then provided to the cancerous tissue in 8 fields spaced at an angle of 45°.

### Example 6

### Treatment of Liver Tumor

A 56-year old female is diagnosed with a liver tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 28 | 1 | 1 | 1 | 0 | 0 | 0 |
| 26 | 1 | 1 | 0 | 1 | 1 | 0 |
| 27 | 1 | 1 | 0 | 1 | 1 | 1 |

FLASH-RT treatment is then provided to the cancerous tissue in 3 fields spaced at an angle of 120°.

### Example 7

### Treatment of Esophageal Tumor

A 51-year old male is diagnosed with an esophageal tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 18 | 1 | 0 | 0 | 1 | 0 | 1 |
| 19 | 1 | 0 | 0 | 1 | 1 | 0 |
| 20 | 1 | 0 | 0 | 1 | 1 | 1 |
| 21 | 1 | 1 | 0 | 0 | 0 | 0 |
| 22 | 1 | 1 | 0 | 0 | 0 | 1 |

FLASH-RT treatment is then provided to the cancerous tissue in 5 fields spaced at an angle of 72°.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and it is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

### DISCLOSED EMBODIMENTS

Embodiment 1) A method for supplying a field of ionizing radiation to a target tissue comprising:
providing an ionizing radiation;
forming at least two fields of shifted and compensated ionizing radiation by;
shifting the range of the ionizing radiation by passing the ionizing radiation through an adjustable range shifter, so that the Bragg peak of the ionizing radiation coincides with the target tissue;
compensating the range of the ionizing radiation by passing the ionizing radiation through an adjustable range compensator, so that the Bragg peak of the ionizing radiation coincides with the target tissue; and
directing to the target tissue at least two fields of the shifted and compensated ionizing radiation to provide a uniform dose distribution across a target volume.

Embodiment 2) The method of embodiment 1, wherein said at least two fields of the shifted and compensated ionizing radiation are directed in a dose rate of at least 40 Gy/s.

Embodiment 3) The method of any preceding embodiment, wherein said at least two fields of the shifted and compensated ionizing radiation does not substantially extend proximally beyond a distal edge of the target location.

Embodiment 4) The method of any preceding embodiment, wherein said at least two fields of the shifted and compensated ionizing radiation comprises protons, helium, carbon, argon or neon.

Embodiment 5) The method of any preceding embodiment wherein said at least two fields of the shifted and compensated ionizing radiation comprises protons.

Embodiment 6) The method of any preceding embodiment, wherein said target location comprises cancerous tissue.

Embodiment 7) The method of any preceding embodiment, wherein said range shifter comprises multiple plates that reduce the range of the ionizing radiation, and said combinations of the range shifters are calculated by applying parameters determined using an inverse-planning optimization protocol, wherein said parameters comprise the number and location of the plates through which the ionizing radiation is transmitted.

Embodiment 8) The method of any preceding embodiment, wherein said range compensator contours are calculated by applying parameters determined using an inverse-planning optimization protocol.

Embodiment 9) The method of embodiment 8, wherein said inverse-planning optimization determines the distribution parameters of the ionizing radiation.

Embodiment 10) The method of embodiment 8 or 9, wherein said inverse-planning optimization determines the weighting parameters of the ionizing radiation.

Embodiment 11) The method of any preceding embodiment, wherein said at least two fields of the shifted and compensated ionizing radiation comprises three fields of the shifted and compensated ionizing radiation.

Embodiment 12) The method of any preceding embodiment, wherein said at least two fields of the shifted and compensated ionizing radiation comprises four fields of the shifted and compensated ionizing radiation.

Embodiment 13) The method of any preceding embodiment, wherein said at least two fields of the shifted and compensated ionizing radiation comprises five fields of the shifted and compensated ionizing radiation.

Embodiment 14) A system for administering at least two fields of shifted and compensated ionizing radiation to a target tissue comprising:
an ionizing radiation source configured to produce a charged particle beam;
a universal range shifter adjusted to shift the range of the charged particle beam so that the Bragg peak of the charged particle beam coincides with the target tissue; and
a range compensator adjusted to compensate the range of the charged particle beam so that the Bragg peak of the ionizing radiation coincides with the contour of the target tissue.

Embodiment 15) The system of embodiment 14, wherein said fields are applied in a dose rate of at least 40 Gy/s.

Embodiment 16) The system of embodiments 14-15, wherein said fields do not substantially extend proximally beyond a distal edge of the target tissue.

Embodiment 17) The system of embodiments 14-16, wherein said target tissue comprises a neoplasm or benign tumor.

Embodiment 18) The system of embodiments 14-17, wherein said range shifter comprises multiple plates that reduce the range of the ionizing radiation, and said combinations of the range shifters are calculated by applying parameters determined using an inverse-planning optimization protocol, wherein said parameters comprise the number and location of the plates through which the ionizing radiation is transmitted.

Embodiment 19) The system of embodiments 14-18, wherein said range compensator contours are calculated by applying parameters determined using an inverse-planning optimization protocol.

Embodiment 20) The system of embodiments 14-19, wherein said at least two fields of the shifted and compensated ionizing radiation comprises three fields.

Embodiment 21) The system of embodiments 14-29, wherein said at least two fields of the shifted and compensated ionizing radiation comprises four fields.

Embodiment 22) The system of embodiments 14-21, wherein said at least two fields of the shifted and compensated ionizing radiation comprises five fields.

Embodiment 23) A system for producing at least two shifted, compensated fields of particle beams at a dose rate of at least 40 Gy/s, said system comprising:
an ionizing radiation source configured to produce a particle beam;
a universal range shifter configured to adjustably shift the range of the particle beam; and
a range compensator configured to adjustably compensate the range of the particle beam.

Embodiment 24) The system of embodiment 23, wherein said particle beams comprise protons, helium, carbon, argon, or neon.

Embodiment 25) A method of treating a target tissue, comprising;
diagnosing a target tissue;
mapping the target tissue;
developing a radiotherapy treatment plan to administer an effective amount of shifted and compensated ionizing radiation to the target tissue;
shifting and compensating an ionizing radiation using a system comprising;
   an ionizing radiation source configured to produce a particle beam;
   a universal range shifter adjusted to shift the range of the proton/particle beam so that the Bragg peak of the particle beam coincides with the target tissue;
   a range compensator adjusted to compensate the range of the particle beam so that the Bragg peak of the particle beam coincides with the contour of the target tissue; and
   administering the particle beam to the target tissue.

Embodiment 26) The method of embodiment 25, wherein said particle beam is applied in a dose rate of at least 40 Gy/s.

Embodiment 27) The method of embodiment 25, wherein said target tissue comprises a neoplasm or benign tumor.

Embodiment 28) The method of embodiment 26, wherein said particle beam does not substantially extend proximally beyond a distal edge of the neoplasm or benign tumor.

Embodiment 29) The method of embodiments 25-28, wherein said range shifter comprises multiple plates that reduce the range of the ionizing radiation, and said range shifter plate positioning is determined by applying parameters calculated using an inverse-planning optimization protocol, wherein said parameters comprise the number and location of the plates through which the ionizing radiation is transmitted.

Embodiment 30) The method of embodiments 25-29, wherein the shape of said range compensator is calculated by applying parameters determined using an inverse-planning optimization protocol.

Embodiment 31) The method of embodiments 25-30, wherein said at least two fields of the shifted and compensated ionizing radiation comprises three fields.

Embodiment 32) The method of embodiments 25-31, wherein said at least two fields of the shifted and compensated ionizing radiation comprises four fields.

Embodiment 33) The method of embodiments 25-32, wherein said at least two fields of the shifted and compensated ionizing radiation comprises five fields.

Embodiment 34) The method of embodiments 25-33, wherein said particle beams comprise protons, helium, carbon, argon, or neon.

Embodiment 35) A method of adjusting a proton therapy device comprising;
receiving a treatment plan designed to apply FLASH-RT to a target location, wherein said treatment plan comprises a target location, a three-dimensional target shape, a number of treatment fields, and target dose rate of at least 40 Gy/s; and
modifying the energy or range of the proton therapy device using a range shifter and a range compensator, wherein said range shifter comprises multiple plates that reduce the range of the ionizing radiation, wherein said modifying comprises use of an inverse-planning protocol to determine range shifter and range compensator parameters so that the Bragg peak of the energy output of the proton therapy device coincides with a target tissue, and wherein said parameters comprise the number and location of the plates through which the energy or range of the proton therapy device is transmitted.

Embodiment 36) The method of embodiment 35, wherein said energy output of the proton therapy device does not substantially extend proximally beyond a distal edge of the cancerous tissue.

Embodiment 37) A universal range shifter comprising six polycarbonate plastic plates, and capable of producing a range shifted ionizing radiation by reducing the range of an ionizing radiation between 0 cm to 34 cm in 1 cm increments.

Embodiment 38) The universal range shifter of embodiment 37, wherein said ionizing radiation comprises protons, helium, carbon, argon, or neon.

Embodiment 39) The universal range shifter of embodiment 38, wherein said ionizing radiation comprises protons.

Embodiment 40) The universal range shifter of embodiment 37, wherein the thickness of said polycarbonate plastic plates is 1, 2, 3, 7, 7, and 14 cm WET.

Embodiment 41) A radiotherapy treatment device comprising a universal range shifter, said universal range shifter comprising six polycarbonate plastic plates, and capable of producing a range shifted ionizing radiation by reducing the range of an ionizing radiation between 0 cm to 34 cm in 1 cm increments.

Embodiment 42) The radiotherapy therapy treatment device of embodiment 41, wherein said ionizing radiation comprises protons or other ions.

Embodiment 43) The radiotherapy therapy treatment device of embodiment 42, wherein said ionizing radiation comprises protons.

Embodiment 44) The radiotherapy treatment device of embodiment 43, wherein the thickness of said polycarbonate plastic plates is 1, 2, 3, 7, 7, and 14 cm WET.

Embodiment 45) A method of reducing the range of an ionizing radiation by between 1 cm and 34 cm in 1 cm increments, said method comprising passing the ionizing radiation through a universal range shifter comprising six polycarbonate plastic plates.

Embodiment 46) The method of embodiment 45, wherein said ionizing radiation comprises protons, helium, carbon, argon, or neon.

Embodiment 47) The method of embodiment 45, wherein said ionizing radiation comprises protons.

Embodiment 48) The method of embodiment 47, wherein the thickness of said polycarbonate plastic plates is 1, 2, 3, 7, 7, and 14 cm WET.

Embodiment 49) A method for treating a cancerous tissue comprising;
providing an ionizing radiation transmission beam with a dose rate of at least 40 Gy/s;
adjusting the energy or range of the ionizing radiation transmission beam such that the Bragg peak of the beam coincides with a point between 3 mm and 5 mm from an edge of the cancerous tissue; and
applying the ionizing radiation transmission beam to the cancerous tissue.

Embodiment 50) The method of embodiment 49, wherein said ionizing radiation transmission beam comprises protons, helium, carbon, argon, or neon.

Embodiment 51) The method of embodiment 50, wherein said ionizing radiation comprises protons.

1. A method for supplying a field of ionizing radiation to a target tissue comprising:
providing an ionizing radiation;
forming at least two fields of shifted and compensated ionizing radiation by;
shifting the range of the ionizing radiation by passing the ionizing radiation through an adjustable range shifter, so that the Bragg peak of the ionizing radiation coincides with the target tissue;
compensating the range of the ionizing radiation by passing the ionizing radiation through an adjustable range compensator, so that the Bragg peak of the ionizing radiation coincides with the target tissue; and
directing to the target tissue at least two fields of the shifted and compensated ionizing radiation to provide a uniform dose distribution across a target volume.

2. The method of claim 1, wherein said at least two fields of the shifted and compensated ionizing radiation are directed in a dose rate of at least 40 Gy/s.

3. The method of claim 2, wherein said at least two fields of the shifted and compensated ionizing radiation does not substantially extend proximally beyond a distal edge of the target location.

4. The method of claim 3, wherein said at least two fields of the shifted and compensated ionizing radiation comprises protons, helium, carbon, argon or neon.

5. The method of claim 4, wherein said at least two fields of the shifted and compensated ionizing radiation comprises protons.

6. The method of claim 5, wherein said target location comprises cancerous tissue.

7. The method of claim 6, wherein said range shifter comprises multiple plates that reduce the range of the ionizing radiation, and said combinations of the range shifters are calculated by applying parameters determined using an inverse-planning optimization protocol, wherein said parameters comprise the number and location of the plates through which the ionizing radiation is transmitted.

8. The method of claim 7, wherein said range compensator contours are calculated by applying parameters determined using an inverse-planning optimization protocol.

9. The method of claim 8, wherein said inverse-planning optimization determines the distribution parameters of the ionizing radiation.

10. The method of claim 9, wherein said inverse-planning optimization determines the weighting parameters of the ionizing radiation.

11. The method of claim 10, wherein said at least two fields of the shifted and compensated ionizing radiation comprises three fields of the shifted and compensated ionizing radiation.

12. The method of claim 11, wherein said at least two fields of the shifted and compensated ionizing radiation comprises four fields of the shifted and compensated ionizing radiation.

13. The method of claim 12, wherein said at least two fields of the shifted and compensated ionizing radiation comprises five fields of the shifted and compensated ionizing radiation.

14. A system for administering at least two fields of shifted and compensated ionizing radiation to a target tissue comprising:
an ionizing radiation source configured to produce a charged particle beam;
a universal range shifter adjusted to shift the range of the charged particle beam so that the Bragg peak of the charged particle beam coincides with the target tissue; and
a range compensator adjusted to compensate the range of the charged particle beam so that the Bragg peak of the ionizing radiation coincides with the contour of the target tissue.

15. The system of claim 14, wherein said fields are applied in a dose rate of at least 40 Gy/s.

16. The system of claim 15, wherein said fields do not substantially extend proximally beyond a distal edge of the target tissue.

17. The system of claim 16, wherein said target tissue comprises a neoplasm or benign tumor.

18. The system of claim 17, wherein said range shifter comprises multiple plates that reduce the range of the ionizing radiation, and said combinations of the range shifters are calculated by applying parameters determined using an inverse-planning optimization protocol, wherein said parameters comprise the number and location of the plates through which the ionizing radiation is transmitted.

19. The system of claim 18, wherein said range compensator contours are calculated by applying parameters determined using an inverse-planning optimization protocol.

20. The system of claim 19, wherein said at least two fields of the shifted and compensated ionizing radiation comprises three fields.
